(19) United States
(12) Patent Application Publication
(10) Pub. No.: US 2005/0119288 A1
(43) Pub. Date: Jun. 2, 2005

(54) Dosing schedule for a novel anticancer agent

(75) Inventors: Samit Kumar Bhattacharya, Groton, CT (US); Richard Damian Connell, East Lyme, CT (US); Jitesh Jani, East Lyme, CT (US); James Dale Moyer, East Lyme, CT (US); Dennis A. Noe, Madison, CT (US); Stefanus Johannes Steyn, Oakdale, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/919,831

(22) Filed: Aug. 17, 2004

Related U.S. Application Data
(60) Provisional application No. 60/495,919, filed on Aug. 18, 2003.

(51) Int. Cl. A61K 31/517

(52) U.S. Cl. 514/266.2; 514/266.22; 514/266.4

(57) ABSTRACT

The invention is directed to methods for the a method for treating overexpression of the erbB2 in a mammal in need of treatment by administering to the mammal a therapeutically effective amount of a first inhibitor of an erbB2 receptor and then, after an interval of less than 24 hours, administering to the mammal from one to six therapeutically effective amounts of the same or different inhibitor of the erbB2 receptor. The invention is also directed to a slow daily infusion of the erbB2 inhibitor. The overexpression of the erbB2 receptor can result in abnormal cell growth and lead to cancer. By the methods of the invention, the efficacy and safety of the inhibitors is increased. The invention is also directed to kits for facilitating the dose administration method of the invention.

### CROSS-REFERENCE TO RELATED APPLICATION

Reference is made to U.S. Provisional Application Ser. No. 60/495,919, filed Aug. 18, 2003, the disclosure of which is incorporated herein by reference in its entirety.

### FIELD OF THE INVENTION

The invention is directed generally to methods of drug administration. More particularly, the invention relates to administration of anticancer agents including inhibitors of erbB2 receptor. This invention also relates to methods for improved administration of inhibitors of protein receptor tyrosine kinases that are useful in the treatment of abnormal cell growth, such as cancer, in mammals. This invention also relates to kits useful in the administration of using such inhibitors in the treatment of abnormal cell growth in mammals, especially humans.

### BACKGROUND OF THE INVENTION

It is known that a cell may become cancerous by virtue of the transformation of a portion of its DNA into an oncogene which is a gene that on activation, leads to the formation of malignant tumor cells. Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. Moreover some receptor tyrosine kinases are substrates for the same or other protein kinases, a process that may regulate kinase function. Receptor tyrosine kinases are classified in families, one of which is the erb family, including erbB1, and erbB2. It is known that kinases such as erbB2 are frequently aberrantly expressed in common human cancers such as breast cancer, gastrointestinal cancer such as colon, rectal or stomach cancer, leukemia, and ovarian, bronchial or pancreatic cancer. It has also been shown that epidermal growth factor receptor (erbB1), which possesses tyrosine kinase activity, is mutated and/or overexpressed in many human cancers such as brain, lung, squamous cell, bladder, gastric, breast, head and neck, oesophageal, gynecological and thyroid tumors. Accordingly, it has been recognized that inhibitors of receptor tyrosine kinases are useful as selective inhibitors of the growth of mammalian cancer cells. Abnormal cell growth can be associated with the cellular expression of erb receptors.

However, it has not been sufficiently appreciated that the method of inhibitor administration can affect the efficacy of the inhibitor.

### SUMMARY OF THE INVENTION

The invention is directed generally to methods and kits for inhibition of abnormal cell growth. More particularly, the invention relates to improved dosing schedules for anti-cancer agents.

The present invention relates to a method for treating overexpression of the erbB2 receptor in a mammal in need of such treatment, said method comprising:
(a) administering to said mammal a therapeutically effective amount of a first inhibitor of the erbB2 receptor; and
(b) subsequently administering to said mammal, after an interval comprising less than 24 hours, from one to six therapeutically effective amounts of a second inhibitor of the erbB2 receptor.

In one preferred embodiment of the present invention one to four therapeutically effective amounts of said second inhibitor of the erbB2 receptor can be administered in step (b) of said method. In a more preferred embodiment one to two therapeutically effective amounts of said second inhibitor of the erbB2 receptor are administered in step (b) of said method. In another embodiment, one therapeutically effective amount of said second inhibitor of the erbB2 receptor is administered in step (b) of said method.

In another embodiment of the present invention the interval in step (b) of said method is less than 12 hours. In a preferred embodiment the interval in step (b) of said method is less than 6 hours. In a more preferred embodiment the interval in step (b) of said method is less than 3 hours. In most preferred embodiment the interval in step (b) of said method is less than 1 hour.

The administration of the inhibitor in steps (a) and (b) can comprise orally, buccally, sublingually, intranasally, intragastrically, intraduodenally, topically, intraocularly, rectally, or vaginally.

In one embodiment of the invention, the first inhibitor in step (a) is the same as the second inhibitor in step (b). In one embodiment of the present method the first amount can differ from the subsequent one to six amounts. In another embodiment of the present invention the inhibitor in (a) can be other than the inhibitor in (b). In one particular embodiment, the inhibitor in (a) is the same as the inhibitor in (b), optionally the same stereoisomer or same salt form. In another embodiment of the treatment, the first inhibitor in (a) is synergistic with the second inhibitor in (b). The first inhibitor in (a), the second inhibitor in (b), or both, can be an antagonist of the erbB2 receptor.

In one embodiment of the present invention the therapeutically effective amount of said first inhibitor of the erbB2 receptor differs from the one to six therapeutically effective amounts of said second inhibitor of the erbB2 receptor. In one preferred embodiment of the present invention the first inhibitor in (a) is other than the second inhibitor in (b). In another preferred embodiment the first inhibitor in (a) is synergistic with the second inhibitor in (b). In another preferred embodiment of the present invention the first inhibitor in (a), the second inhibitor in (b), or both, are an antagonist of the erbB2 receptor.

In one preferred embodiment of the present invention the first inhibitor in (a), the second inhibitor in (b), are independently selected from small molecules and monoclonal antibodies. In one preferred embodiment both the first inhibitor in (a), the second inhibitor in (b), are small molecules or monoclonal antibodies. In another preferred embodiment of the present invention the first inhibitor in (a), the second inhibitor in (b), or both are selective for erbB2 receptors.

The method of treatment of the invention can further comprise that the inhibitor in (a), the inhibitor in (b), or both, have an in vivo half life of between half an hour and eight hours.

The method of the invention can comprise administration of an inhibitor wherein the inhibitor in (a), the inhibitor in (b), or both, are other than substantially cytotoxic.

The method can comprise administration of an inhibitor wherein the inhibitor in (a), the inhibitor in (b), or both, are other than substantially a mitosis inhibitor.

In one aspect of the invention, the administration is controlled release. The controlled release formulation can be administered orally, buccally, sublingually, intranasally, intragastrically, intraduodenally, topically, intraocularly, rectally, or vaginally.

In one embodiment of the method of the invention, the inhibitor in (a) and the inhibitor in (b) are independently selected from small molecules and monoclonal antibodies. In one preferred embodiment both the inhibitor in (a) and the inhibitor in (b) are small molecules or monoclonal antibodies. The small molecule can be less than 4,000 Daltons.

The first inhibitor in (a), the second inhibitor in (b), or both, can be selective for erbB2 receptors.

In yet another embodiment of the treatment, the first inhibitor in (a), the second inhibitor in (b), or both comprise a compound of the formula 1: 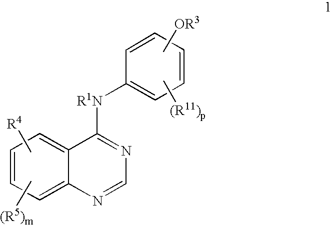
or a pharmaceutically acceptable salt, solvate or prodrug thereof.

In formula 1 m is an integer from 0 to 3;
p is an integer from 0 to 4;
each R¹ and R² is independently selected from H and C₁-C₆ alkyl;
R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R⁸ groups;
R⁴ is —(CR¹⁶R¹⁷)ₘ—C═C—(CR¹⁶R¹⁷)ₜR⁹, —(C R¹⁶R¹⁷)ₘ—C═C—(CR¹⁶R¹⁷)ₜ—R⁹, —(CR¹⁶R¹⁷)ₘ—C═C—(CR¹⁶R¹⁷)ₖR¹³, —(CR¹⁶R¹⁷)ₘ—C═C—(CR¹⁶R¹⁷)ₖR¹³, or —(CR¹⁶R¹⁷)ₜR⁹, wherein the attachment point to R⁹ is through a carbon atom of the R⁹ group, each k is an integer from 1 to 3, each t is an integer from 0 to 5, and each m is an integer from 0 to 3;
each R⁵ is independently selected from halo, hydroxy, —NR¹R², C₁-C₆ alkyl, trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;
each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo(═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₆ alkenyl, C₂-C₆ alkynyl, hydroxy, and C₁-C₆ alkoxy;
or R⁶ and R⁷, or R^{6a} and R⁷, when attached to the same nitrogen atom, can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;
each R⁸ is independently selected from oxo(═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo(═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
R⁹ is a non-aromatic mono-cyclic ring, a fused or bridged bicyclic ring, or a spirocyclic ring, wherein said ring contains from 3 to 12 carbon atoms wherein from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;
each R¹¹ is independently selected from the substituents provided in the definition of R⁸, except R¹¹ is not oxo(═O);
R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷—NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2;
R¹³ is —NR¹R¹⁴ or —OR¹⁴;
R¹⁴ is H, R¹⁵, —C(O)R¹⁵, —SO₂R¹⁵, —C(O)NR¹⁵R⁷, —SO₂NR¹⁵R⁷, or —CO₂R¹⁵;
R¹⁵ is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo(═O) moiety, and the aryl and heterocyclic moieties of the foregoing R¹⁵ groups are optionally substituted with 1 to 3 R⁸ substituents;
each R¹⁶ and R¹⁷ is independently selected from H, C₁-C₆ alkyl, and —CH₂OH, or R¹⁶ and R¹⁷ are taken together as —CH₂CH₂— or —CH₂CH₂CH₂—;
R¹⁸ is C₁-C₆ alkyl wherein each carbon not bound to a N or O atom, or to S(O)ⱼ, wherein j is an integer from 0 to 2, is optionally substituted with R¹²;
and wherein any of the above-mentioned substituents comprising a CH₃ (methyl), CH₂ (methylene), or CH (methine) group, which is not attached to a halogeno, SO or SO₂ group or to a N, O or S atom, is optionally substituted with a group selected from hydroxy, halo, C₁-C₄ alkyl, C₁-C₄ alkoxy and —NR¹R².

The term “halo”, as used herein, unless otherwise indicated, includes fluoro, chloro, bromo or iodo. Preferred halo groups are fluoro and chloro.

The term “alkyl”, as used herein, unless otherwise indicated, includes saturated monovalent hydrocarbon radicals having straight, cyclic (including mono- or multi-cyclic moieties) or branched moieties. It is understood that for said alkyl group to include cyclic moieties it must contain at least three carbon atoms.

The term “cycloalkyl”, as used herein, unless otherwise indicated, includes saturated monovalent hydrocarbon radicals having cyclic (including mono- or multi-cyclic) moieties.

The term “alkenyl”, as used herein, unless otherwise indicated, includes alkyl groups, as defined above, having at least one carbon-carbon double bond.

The term “alkynyl”, as used herein, unless otherwise indicated, includes alkyl groups, as defined above, having at least one carbon-carbon triple bond.

The term “aryl”, as used herein, unless otherwise indicated, includes an organic radical derived from an aromatic hydrocarbon by removal of one hydrogen, such as phenyl or naphthyl.

The term “alkoxy”, as used herein, unless otherwise indicated, includes —O-alkyl groups wherein alkyl is as defined above.

The term “4 to 10 membered heterocyclic”, as used herein, unless otherwise indicated, includes aromatic and non-aromatic heterocyclic groups containing one or more heteroatoms each selected from O, S and N, wherein each heterocyclic group has from 4 to 10 atoms in its ring system. Non-aromatic heterocyclic groups include groups having only 4 atoms in their ring system, but aromatic heterocyclic groups must have at least 5 atoms in their ring system. The heterocyclic groups include benzo-fused ring systems and ring systems substituted with one or more oxo moieties. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

The term “Me” means methyl, “Et” means ethyl, and “Ac” means acetyl.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

The method of treatment of the invention can include administration of an erbB2 receptor inhibitor wherein the inhibitor in (a), the inhibitor in (b), or both, comprise a compound selected from the group consisting of gefitinib (IRESSA, ZD1839), trastuzumab, cetuximab, erlotinib, IDM-1, ABX-EGF, canertinib hydrochloride, EGF-P64k vaccine, EKB-569, EMD-72000, GW-572016, MDX-210, ME-103, YMB-1001, 2C4 antibody, APC-8024, CP-724714, E75, Her-2/neu vaccine, Herzyme, TAK-165, ADL-681, B-17, D-69491, Dab-720, EGFrvill, EHT-102, FD-137, HER-1 vaccine, HuMax-DGFr, ME-104, MR¹-1, SC-100, trastuzumab-DM1, YMB-1005, AEE-788 (Novartis), mTOR inhibitors, including Rapamycin (Rapamune, Siolimus, Wyeth), CCI-779 (Wyeth), AP23573 (ARIAD) and RAD001 (Novartis).

In one embodiment of the present invention the overexpression of the erbB2 receptor is determined using a cytogenetic test, a measurement of fluorescence in-situ hybridization, an immunohistochemistry test, a flow cytometric test, a test based on reverse transcriptase polymerase chain reaction, or any combination thereof.

In one embodiment of the present invention the mammal is a human and the abnormal cell growth is a cancer. The mammal can also be an experimental animal, a household pet, a barnyard animal, or any other mammal.

The method of treatment of the invention can further comprise achieving plasma levels of the first inhibitor in (a), the second inhibitor in (b), or both, between 10 ng/ml and 4000 ng/ml.

In one embodiment of the invention, the first inhibitor in (a) and the second inhibitor in (b) are each independently selected from the group consisting of:
(±)-(3-Methyl-4-(pyridin-3-yloxy)-phenyl)-(6-piperidin-3-ylethynyl-quinazolin-4-yl)-amine;
(±)-(3-Methyl-4-(pyridin-3-yloxy)-phenyl)-(6-piperidin-3-ylethynyl-quinazolin-4-yl)-amine;
(−)-(3-Methyl-4-(pyridin-3-yloxy)-phenyl)-(6-piperidin-3-ylethynyl-quinazolin-4-yl)-amine;
2-Methoxy-N-(3-{4-(3-methyl-4-(pyridin-3-yloxy)-phenylamino)-quinazolin-6-yl}-prop-2-ynyl)-acetamide;
(±)-(3-Methyl-4-(6-methyl-pyridin-3-yloxy)-phenyl)-(6-piperidin-3-ylethynyl-quinazolin-4-yl)-amine;
(±)-(3-Methyl-4-(6-methyl-pyridin-3-yloxy)-phenyl)-(6-piperidin-3-ylethynyl-quinazolin-4-yl)-amine;
(−)-(3-Methyl-4-(6-methyl-pyridin-3-yloxy)-phenyl)-(6-piperidin-3-ylethynyl-quinazolin-4-yl)-amine;
2-Methoxy-N-(3-{4-(3-methyl-4-(2-methyl-pyridin-3-yloxy)-phenylamino)-quinazolin-6-yl}-prop-2-ynyl)-acetamide;
(3-Methyl-4-(2-methyl-pyridin-3-yloxy)-phenyl)-(6-piperidin-4-ylethynyl-quinazolin-4-yl)-amine;
(3-Methyl-4-(6-methyl-pyridin-3-yloxy)-phenyl)-(6-piperidin-4-ylethynyl-quinazolin-4-yl)-amine;
2-Methoxy-N-(3-{4-(3-methyl-4-(6-methyl-pyridin-3-yloxy)-phenylamino)-quinazolin-6-yl}-prop-2-ynyl)-acetamide;
2-Fluoro-N-(3-{4-(3-methyl-4-(6-methyl-pyridin-3-yloxy)-phenylamino)-quinazolin-6-yl}-prop-2-ynyl)-acetamide;
E-2-Methoxy-N-(3-{4-(3-methyl-4-(6-methyl-pyridin-3-yloxy)-phenylamino)-quinazolin-6-yl}-allyl)-acetamide;
(3-Methyl-4-(pyridin-3-yloxy)-phenyl)-(6-piperidin-4-ylethynyl-quinazolin-4-yl)-amine;
2-Methoxy-N-(1-{4-(3-methyl-4-(6-methyl-pyridin-3-yloxy)-phenylamino)-quinazolin-6-ylethynyl}-cyclopropyl)-acetamide;
E-N-(3-{4-(3-Chloro-4-(6-methyl-pyridin-3-yloxy)-phenylamino)-quinazolin-6-yl}-allyl)-2-methoxy-acetamide;
N-(3-{4-(3-Chloro-4-(6-methyl-pyridin-3-yloxy)-phenylamino)-quinazolin-6-yl}-prop-2-ynyl)-acetamide;
N-(3-{4-(3-Methyl-4-(6-methyl-pyridin-3-yloxy)-phenylamino)-quinazolin-6-yl}-prop-2-ynyl)-acetamide;
E-N-(3-{4-(3-Chloro-4-(6-methyl-pyridin-3-yloxy)-phenylamino)-quinazolin-6-yl}-allyl)-acetamide;
E-2-Ethoxy-N-(3-{4-(3-methyl-4-(6-methyl-pyridin-3-yloxy)-phenylamino)-quinazolin-6-yl}-allyl)-acetamide;
1-Ethyl-3-(3-{4-(3-methyl-4-(6-methyl-pyridin-3-yloxy)-phenylamino)-quinazolin-6-yl}-prop-2-ynyl)-urea;
Piperazine-1-carboxylic acid (3-{4-(3-methyl-4-(6-methyl-pyridin-3-yloxy)-phenylamino)-quinazolin-6-yl}-prop-2-ynyl)-amide;
(±)-2-Hydroxymethyl-pyrrolidine-1-carboxylic acid (3-{4-(3-methyl-4-(6-methyl-pyridin-3-yloxy)-phenylamino)-quinazolin-6-yl}-prop-2-ynyl)-amide;
(±)-2-Hydroxymethyl-pyrrolidine-1-carboxylic acid (3-{4-(3-methyl-4-(6-methyl-pyridin-3-yloxy)-phenylamino)-quinazolin-6-yl}-prop-2-ynyl)-amide;
(−)-2-Hydroxymethyl-pyrrolidine-1-carboxylic acid (3-{4-(3-methyl-4-(6-methyl-pyridin-3-yloxy)-phenylamino)-quinazolin-6-yl}-prop-2-ynyl)-amide;
2-Dimethylamino-N-(3-{4-(3-methyl-4-(pyridin-3-yloxy)-phenylamino)-quinazolin-6-yl}-prop-2-ynyl)-acetamide;
E-N-(3-{4-(3-Methyl-4-(6-methyl-pyridin-3-yloxy)-phenylamino)-quinazolin-6-yl}-allyl)-methanesulfonamide;
Isoxazole-5-carboxylic acid (3-{4-(3-methyl-4-(6-methyl-pyridin-3-yloxy)-phenylamino)-quinazolin-6-yl}-prop-2-ynyl)-amide;
1-(1,1-Dimethyl-3-{4-(3-methyl-4-(6-methyl-pyridin-3-yloxy)-phenylamino)-quinazolin-6-yl}-prop-2-ynyl)-3-ethyl-urea;

The method of treatment includes use of a single agent that inhibits an erbB2 receptor, as well as use of two different agents. The single agent and at least one of the two agents is preferably an agent according to Formula 1. Thus, in one embodiment, the inhibitor is selected from the group consisting of (±)-(3-methyl-4-(6-methyl-pyridin-3-yloxy)-phenyl)-(6-piperidin-3-ylethynyl-quinazolin-4-yl)-amine; and pharmaceutically acceptable salts, prodrugs and solvates thereof. In another embodiment, the inhibitor is selected from the group consisting of (3-methyl-4-(6-methyl-pyridin-3-yloxy)-phenyl)-(6-piperidin-4-ylethynyl-quinazolin-4-yl)-amine; and pharmaceutically acceptable salts, prodrugs and solvates thereof. In yet another embodiment, the inhibitor is selected from the group consisting of: E-2-methoxy-N-(3-{4-(3-methyl-4-(6-methyl-pyridin-3-yloxy)-phenylamino)-quinazolin-6-yl}-allyl)-acetamide; and pharmaceutically acceptable salts, prodrugs and solvates thereof. In still another embodiment, the inhibitor is selected from the group consisting of E-N-(3-{4-(3-chloro-4-(6-methyl-pyridin-3-yloxy)-phenylamino)-quinazolin-6-yl}-allyl)-2-methoxy-acetamide; and pharmaceutically acceptable salts, prodrugs and solvates thereof. In still yet another embodiment, the inhibitor is selected from the group consisting of: E-N-(3-{4-(3-chloro-4-(6-methyl-pyridin-3-yloxy)-phenylamino)-quinazolin-6-yl}-allyl)-acetamide; and pharmaceutically acceptable salts, prodrugs and solvates thereof. In a particular embodiment of the invention, the inhibitor is selected from the group consisting of piperazine-1-carboxylic acid (3-{4-(3-methyl-4-(6-methyl-pyridin-3-yloxy)-phenylamino)-quinazolin-6-yl}-prop-2-ynyl)-amide; and pharmaceutically acceptable salts, prodrugs and solvates thereof. In another particular embodiment of the invention, the inhibitor is selected from the group consisting of E-N-(3-{4-(3-methyl-4-(6-methyl-pyridin-3-yloxy)-phenylamino)-quinazolin-6-yl}-allyl)-methanesulfonamide; and pharmaceutically acceptable salts, prodrugs and solvates thereof. In another aspect of the invention, the first inhibitor of (a), the second inhibitor of (b), or both, are in a pharmaceutically acceptable carrier.

In one embodiment of the present invention overexpression of the erbB2 receptor results in abnormal cell growth. The abnormal cell growth that is treated with the first and second erbB2 receptor inhibitors may be cancer. The cancer can be selected from the group consisting of acral lentiginous melanoma, an actinic keratosis, adenocarcinoma, adenoid cystic carcinoma, an adenoma, adenosarcoma, adenosquamous carcinoma, an astrocytic tumor, bartholin gland carcinoma, basal cell carcinoma, a bronchial gland carcinoma, capillary carcinoma, a carcinoid, carcinoma, carcinosarcoma, cavernous carcinoma, cholangiocarcinoma, chondosarcoma, choriod plexus papilloma, choriod plexus carcinoma, clear cell carcinoma, cystadenoma, endodermal sinus tumor, endometrial hyperplasia, endometrial stromal sarcoma, endometrioid adenocarcinoma, ependymal carcinoma, epitheloid carcinoma, Ewing's sarcoma, fibrolamellar, focal nodular hyperplasia, gastrinoma, a germ cell tumor, glioblastoma, glucagonoma, hemangiblastoma, hemangioendothelioma, a hemangioma, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, a lentigo maligna melanoma, malignant melanoma, a malignant mesothelial tumor, medulloblastoma, medulloepithelioma, melanoma, meningeal, mesothelial, metastatic carcinoma, mucoepidermoid carcinoma, neuroblastoma, neuroepithelial adenocarcinoma, nodular melanoma, oat cell carcinoma, oligodendroglial, osteosarcoma, pancreatic polypeptide, papillary serous adenocarcinoma, pineal cell, a pituitary tumor, plasmacytoma, pseudosarcoma, pulmonary blastoma, renal cell carcinoma, retinoblastoma, rhabdomyosarcoma, sarcoma, serous carcinoma, small cell carcinoma, a soft tissue carcinoma, somatostatin-secreting tumor, squamous carcinoma, squamous cell carcinoma, submesothelial, superficial spreading melanoma, undifferentiated carcinoma, uveal melanoma, verrucous carcinoma, vipoma, a well differentiated carcinoma, bronchioloalveolar cell carcinoma (BAC) and Wilm's tumor.

In one embodiment the abnormal cell growth is a tumor is selected from the group consisting of a lung, a breast, a skin, a stomach, an intestine, an esophagus, a pancreas, a liver, a bladder, a head, a neck, a brain, a cervical, and an ovary tumor. In one preferred embodiment, the abnormal cell growth is a tumor selected from the group consisting of a breast, a stomach, a pancreas, and an ovary. In a more preferred embodiment, the abnormal cell growth is a breast cancer.

In another embodiment of the invention, the erbB2 receptor inhibitor can be selective for the erbB2 receptor. The method of the invention can further comprise: (c) calculating the ratio of a binding affinity of the inhibitor for the erbB2 receptor and a second binding affinity of the inhibitor for an erbB1 receptor and (d) using the ratio to evaluate selectivity. In one embodiment, the inhibitor is at least two-fold selective for the erbB2 receptor. In another embodiment, the inhibitor is at least ten-fold selective for the erbB2 receptor.

In another embodiment of the present invention relates to a method of treating a subject having abnormal cell growth comprising orally, buccally, sublingually, intranasally, intraocularly, intragastrically, intraduodenally, topically, rectally, or vaginally administering to said subject in need of treatment for abnormal cell growth, within a twenty-four hour period, a first amount of an inhibitor of an erbB2 receptor, a therapeutically synergistically effective second amount of the inhibitor, and optionally, a third or fourth amount of the inhibitor. The inhibitor can be a selective erbB2 receptor inhibitor.

In another embodiment of the invention the invention comprises a kit for treatment of abnormal cell growth, comprising at least two doses of an inhibitor of an erbB2 receptor, the doses suitable for oral, buccal, sublingual, intranasal, intraocular, intragastric, intraduodenal, topical, rectal, or vaginal administration to a subject, and written instructions to administer the doses at least twice daily to a subject having said abnormal cell growth. Advantageously the written instructions are on a label or a package insert. In one embodiment of the kit, the abnormal cell growth is a tumor selected from the group consisting of a lung, a breast, a skin, a stomach, an intestine, an esophagus, a bladder, a head, a neck, a brain, a cervical, and an ovary tumor.

In another embodiment of the invention the invention comprises a method for treating a tumor in a subject in need thereof, the tumor comprising an erbB2 receptor, comprising administering to said subject a therapeutically effective amount of an erbB2 receptor inhibitor by infusion into said subject over a duration of one to eight hours, such that the infusion is more efficacious than a bolus injection. The infusion can be intravenous, intramuscular, intraperitoneal, or subcutaneous. In one embodiment, the inhibitor can be a compound according to formula 1.

In another embodiment of the invention the invention comprises a method of enhancing the efficacy of an erbB2 receptor inhibitor in a subject in need thereof comprising: (a) determining a reference dose of the erbB2 receptor inhibitor, and (b) dividing the dose to increase the efficacy. The increased efficacy is a form of synergy resulting from dividing the dose. In one embodiment, the dose is divided into from two to six daily doses.

In another embodiment, the reference dose has a side-effect and the divided dose has a diminished side-effect. The inhibitor can be at least about two-fold selective for the erbB2 receptor relative to an erbB1 receptor. In yet another embodiment, the inhibitor is at least ten-fold selective for the erbB2 receptor relative to an erbB1 receptor.

The method of enhancing the efficacy can further comprises the steps (c) calculating the ratio of a binding affinity of the inhibitor for the erbB2 receptor and a second binding affinity of the inhibitor for an erbB1 receptor and (d) using the ratio to evaluate selectivity.

In another embodiment of the invention the invention comprises a method for increasing the efficacy of an inhibitor of an erbB2 receptor comprising administering a daily dose of a therapeutically effective amount of the inhibitor to a patient in need thereof, wherein the daily dose is divided to establish a plasma level of the inhibitor in said patient lower than the therapeutically effective amount of a single daily dose and the efficacy is increased.

In another embodiment of the invention comprises a method for enhancing the safety of administration of an erbB2 receptor inhibitor to a subject in need thereof comprising daily administering to said subject from two to six therapeutically effective amounts of the inhibitor.

In another embodiment of the invention comprises a method of enhancing the safety of administration of an erbB2 receptor inhibitor to a subject in need thereof comprising determining a reference daily dose of the inhibitor having a safety profile and dividing the dose to improve the safety profile.

In another embodiment of the invention comprises a kit for treatment of abnormal cell growth in a subject, comprising a dose of an inhibitor of an erbB2 receptor, the dose suitable for intravenous, intramuscular, intraperitoneal, or subcutaneous infusion, and written instructions to infuse the dose into said subject over a duration of one hour to eight hours. In one embodiment of the kit the abnormal cell growth can involve a tumor selected from the group consisting of a lung, a breast, a skin, a stomach, an intestine, an esophagus, a bladder, a pancreas, a liver, a head, a neck, a brain, a cervical, and an ovary tumor.

In another embodiment of the invention comprises a prophylactic treatment for a subject at risk for developing a tumor comprising administering to said subject an effective amount of a selective inhibitor of an erbB2 receptor at least twice per day. In one embodiment of the prophylactic treatment, the inhibitor can be other than an antibody or fragment thereof.

In another embodiment of the invention comprises a method for increasing the efficacy of an inhibitor of an erbB2 receptor comprising administering a daily dose of a therapeutically effective amount of the inhibitor to a patient in need thereof, wherein the daily dose is divided to establish a plasma level of the inhibitor in said patient lower than the therapeutically effective amount of a single daily dose and the efficacy is increased. In one embodiment, the plasma level is expressed as Cave. In another embodiment, the plasma level is expressed as Cₘₐₓ. The inhibitor can be a selective erbB2 receptor inhibitor. In one embodiment, the inhibitor is other than an antibody or fragment thereof.

In yet another embodiment of the present invention relates to a method for treating a tumor in a subject in need thereof, the tumor comprising an erbB2 receptor, comprising administering to said subject a therapeutically effective amount of an erbB2 receptor inhibitor by infusion into said subject over a duration of one to eight hours, such that the infusion is more efficacious than a bolus injection. By a bolus injection is meant a relatively rapid therapeutic infusion, consistent with the properties of the injection site. The infusion can be intravenous, intramuscular, intraperitoneal, or subcutaneous. The subject of the method can be a human but any mammal is suitable. In one embodiment the tumor is a cancer. The infusion can be characterized by an uneven rate in the method of the invention. For example the rate of administration can increase or decrease during infusion. The inhibitor can be selective for the erbB2 receptor. Moreover, the method can further comprise: calculating the ratio of a binding affinity of the inhibitor for the erbB2 receptor and a second binding affinity of the inhibitor for an erbB1 receptor, and using the ratio to evaluate selectivity. Other methods known in the art are also suitable for evaluating selectivity. In one embodiment, the inhibitor is at least two-fold selective for the erbB2 receptor. In another embodiment, the inhibitor is at least ten-fold selective for the erbB2 receptor. The subject of the treatment method of the invention can be a human. The inhibitor can be an antagonist. In one embodiment, the inhibitor is other than an antibody or fragment thereof. In particular, the inhibitor can be a small molecule. The method of the invention can further comprise that the inhibitor has an in vivo half life of between one half and eight hours.

In one embodiment of the present invention relates to a method for treating overexpression of the erbB2 receptor in a mammal in need of such treatment, said method comprising:
(a) determining the overexpression of the erbB2 receptor using a cytogenetic test, a fluorescence in-situ hybridization, an immunohistochemistry test, a flow cytometric test, a reverse transcriptase polymerase chain reaction, or combination thereof;
(b) administering to said mammal a therapeutically effective amount of a first inhibitor of the erbB2 receptor based upon the overexpression of the erbB2 receptor from step
(a); and
(c) subsequently administering to said mammal, after an interval comprising less than 24 hours, from one to six therapeutically effective amounts of a second inhibitor of the erbB2 receptor based upon the overexpression of the erbB2 receptor from step (a).

The method can include infusion of an inhibitor wherein the inhibitor is other than substantially cytotoxic. The method can also include infusion of an inhibitor wherein the inhibitor is other than substantially a mitosis inhibitor.

The method of treatment by infusion of an inhibitor can further comprise that the infusion is at least 20% more efficacious than the bolus injection.

The method of treatment by infusion can further comprise infusion two or three times daily.

The method of treatment by infusion can further comprise achieving plasma levels of the inhibitor between 10 ng/ml and 4000 ng/ml.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “Cₘₐₓ”, as used herein, unless otherwise indicated, means the maximum concentration of an agent in blood, serum, or plasma after administration of the agent. The agent is typically an erbB2 receptor inhibitor according to Formula 1.

The term “AUC”, as used herein, unless otherwise indicated, means area under the curve, is a measure of the concentration of agent integrated over time.

The term “Cave” or “Cₐᵥₑ”, as used herein, unless otherwise indicated, a measure of the average concentration of agent over a defined time period.

The term “PK”, as used herein, unless otherwise indicated, means pharmacokinetics or the distribution of an agent with time.

The terms “QD” and “BID” as used herein, unless otherwise indicated, means daily and twice daily administration, respectively.

The terms “p.o.” and “i.v.” as used herein, unless otherwise indicated, means oral and intravenous routes of administration, respectively.

The term “PD”, as used herein, unless otherwise indicated, means pharmacodynamics, an analysis of functional consequences of an agent.

The term “selectivity”, as used herein, unless otherwise indicated, means efficacy relative to another agent and is commonly presented as a ratio of inhibition constants (IC values, as, for example IC₅₀). Alternatively, selectivity can be measured as the affinity of the inhibitor for the erbB2 receptor relative to affinity for another receptor, e.g., erbB1. Selectivity can be measured in any conventional way known in the art, including, but not limited to absolute potency, potency relative to another agent, efficacy relative to another agent, and presence or extent of non-erbB2 receptor effects.

The term “inhibiting an erbB2 receptor”, as used herein, unless otherwise indicated, means competitive or non-competitive blocking of binding of an activator, that is an agonist, displacing a bound activator, reducing the affinity constant of an activator, increasing the off-rate of an activator, dissociating a multimeric receptor, aggregating a monomeric receptor, or reducing an intracellular metabolic consequence of receptor activation.

The term “synergy” or “synergistic”, as used herein, unless otherwise indicated, means that the combined effect of the two inhibitors is greater than the sum of the effect of each inhibitor alone.

The term “agonist” as used herein, unless otherwise indicated, means drugs that bind to physiological receptor and mimic the effect of the endogenous regulatory compounds. The term “antagonist” as used herein, unless otherwise indicated, means drugs which bind to a receptor and do not mimic, but interfere with, the binding of the endogenous agonist. Such drugs or compounds, which are themselves devoid of intrinsic regulatory activity, but which produce effects by inhibiting the action of an agonist are termed “antagonist.”

The term “side-effect” as used herein, unless otherwise indicated, means the action or effect of a drug other than the desired effect.

The term “diminished side-effect” as used herein, unless otherwise indicated, means diminish action or effect of a drug other than desired effect.

The term “inhibitor” as used herein, unless otherwise indicated, means a chemical substance that stops activity of an enzyme or receptor.

Those compounds of formula 1 that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The compounds of formula 1 may have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of formula 1 may also exist as tautomers. This invention relates to the use of all such tautomers and mixtures thereof.

The subject invention also includes use of isotopically-labelled compounds, and the pharmaceutically acceptable salts, solvates and prodrugs thereof, which are identical to those recited in formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H ³H, ¹³C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples and Preparations below, by substituting a readily available isotopically labelled reagent for a non-isotopically labelled reagent.

Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

### BRIEF DESCRIPTION OF THE DRAWINGS

FIG. 1 shows the anti-tumor efficacy of an inhibitor, E-2-Methoxy-N-(3-{4-(3-methyl-4-(6-methyl-pyridin-3-yloxy)-phenylamino)-quinazolin-6-yl}-allyl)-acetamide administered PO, QD to mice having FRE/erbB2 tumors. The ordinate is a measure of the tumor growth at day 7, relative to vehicle control.

FIG. 2 shows the anti-tumor efficacy of an inhibitor, E-2-Methoxy-N-(3-{4-(3-methyl-4-(6-methyl-pyridin-3-yloxy)-phenylamino)-quinazolin-6-yl}-allyl)-acetamide administered IV, QD to mice having FRE/erbB2 tumors. The ordinate is a measure of the tumor growth at day 7, relative to vehicle control.

FIG. 3 shows the time course of anti-tumor efficacy of an inhibitor, E-2-Methoxy-N-(3-{4-(3-methyl-4-(6-methyl-pyridin-3-yloxy)-phenylamino)-quinazolin-6-yl}-allyl)-acetamide administered PO and QD to SK-OV-3 tumor bearing nu/nu mice. In FIG. 3, the symbols have the following meanings: circle, vehicle, BID; lozenge, inhibitor at 50 mg/kg, QD; triangle, inhibitor at 100 mg/kg, QD; and square, inhibitor at 200 mg/kg, QD.

FIG. 4 shows the time course of anti-tumor efficacy of an inhibitor, E-2-Methoxy-N-(3-{4-(3-methyl-4-(6-methyl-pyridin-3-yloxy)-phenylamino)-quinazolin-6-yl}-allyl)-acetamide administered PO and BID to SK-OV-3 tumor bearing nu/nu mice. In FIG. 4 the symbols have the following meanings: circle, vehicle, BID; cross, inhibitor at 25 mg/kg BID; diamond, inhibitor at 50 mg/kg, BID; and star, inhibitor at 100 mg/kg, BID.

FIG. 5A shows the antitumor efficacy of an inhibitor, E-2-Methoxy-N-(3-{4-(3-methyl-4-(6-methyl-pyridin-3-yloxy)-phenylamino)-quinazolin-6-yl}-allyl)-acetamide administered to mice bearing BT-474 tumors, illustrating the effect of multiplicity of the doses.

FIG. 5B shows the antitumor efficacy of an inhibitor, E-2-Methoxy-N-(3-{4-(3-methyl-4-(6-methyl-pyridin-3-yloxy)-phenylamino)-quinazolin-6-yl}-allyl)-acetamide administered to mice bearing BT-474 tumors, illustrating the effect of the frequency of the doses.

FIG. 6A shows the antitumor efficacy of an inhibitor, E-2-Methoxy-N-(3-{4-(3-methyl-4-(6-methyl-pyridin-3-yloxy)-phenylamino)-quinazolin-6-yl}-allyl)-acetamide administered QD to mice bearing MDA-MB-453 tumors.

FIG. 6B shows the antitumor efficacy of an inhibitor, E-2-Methoxy-N-(3-{4-(3-methyl-4-(6-methyl-pyridin-3-yloxy)-phenylamino)-quinazolin-6-yl}-allyl)-acetamide administered BID to mice bearing MDA-MB-453 tumors.

### DETAILED DESCRIPTION OF THE INVENTION

The method of the invention can comprise administration of an inhibitor wherein the inhibitor in (a), the inhibitor in (b), or both, are other than substantially cytotoxic. Cytotoxicity can be determined by any means common in the art, including, but not limited to measurement of apoptosis and metabolic functions such as respiration and substrate utilization. By substantially cytotoxic is meant that one skilled in the art would recognize that cytotoxicity is generally found upon administration of the agent to a test animal or upon use in an in vitro assay under conditions and concentrations corresponding to the use of the agent in the invention.

The method can comprise administration of an inhibitor wherein the inhibitor in (a), the inhibitor in (b), or both, are other than substantially a mitosis inhibitor. Mitosis can be determined by any means common in the art, including, but not limited to measurements of mitotic index, DNA content and cell number. By substantially a mitosis inhibitor is meant that one skilled in the art would recognize that diminished mitosis is generally found upon administration of the agent to a test animal or upon use in an in vitro assay under conditions and concentrations corresponding to the use of the agent in the invention.

The in vitro activity of the compounds for use in the methods of the present invention can be determined by the amount phosphorylation inhibition by a test compound relative to a control. Recombinant erbB2 (amino acid residues 675-1255) and EGFR (amino acid residues 668-1211) intracellular domains were expressed in Baculovirus-infected Sf9 cells as GST fusion proteins and purified by affinity chromatography on glutathione sepharose beads. The phosphorylation of poly(Glu, Tyr) was measured as described in J. D. Moyer, E. G. Barbacci, K. K. Iwata, L. Arnold, B. Boman, A. Cunningham, et al., Induction of apoptosis and cell cycle arrest by CP-358,774, an inhibitor of epidermal growth factor receptor tyrosine kinase, Cancer Res. 57 (1997) 4838-4848, except the kinase reaction was performed in 50 μl of 50 mM HEPES, pH 7.4, containing 125 mM sodium chloride, 10 mM magnesium chloride, 0.1 mM sodium orthovanadate, and 1 mM ATP.

Tyrosine Phosphorylation in intact cells may be measured using the following assay. NIH3T3 cells transfected with either human EGFR (B. D. Cohen, D. R. Lowy, J. T. Schiller, Transformation-specific interaction of the bovine papillomavirus E5 oncoprotein with the platelet-derived growth factor receptor transmembrane domain and the epidermal growth factor receptor cytoplasmic domain, J. Virol., 67 (1993) 5303-5311) or a chimeric receptor with EGFR extracellullar domain and erbB2 intracellular domain were seeded in 96 well tissue culture plates in DMEM (F. Fazioli, U. H. Kim, S. G. Rhee, C. J. Molloy, O. Segatto, P. P. DiFiore, The erbB-2 mitogenic signaling pathway: tyrosine phosphorylation of phospholipase C-gamma and GTPase-activating protein does not correlate with erbB-2 mitogenic potency, Mol. Cell. Biol., 11 (1991) 2040-2048).

Inhibitors in DMSO (or DMSO vehicle for controls) were added 24 h after plating and incubated with the cells for 2 h at 37° C. Cells were stimulated with human recombinant EGF (50 ng/ml final concentration) for 15 min at room temperature. Medium was aspirated and cells were fixed for 30 min with 100 μl cold 1:1 ethanol:acetone containing 200 μM Na₃VO₄. Plates were washed with wash buffer (0.5% Tween-20 in PBS) and 100 μl block buffer (3% bovine serum albumin in PBS+200 μM fresh sodium orthovanadate) was added. Plates were further incubated for 1 h at room temp and washed twice with wash buffer. Anti-phosphotyrosine antibody (PY54) labeled with horseradish peroxidase was added to wells and incubated for 1 h at room temp. Antibody was removed by aspiration and plates were washed 4 times with wash buffer. The colorimetric signal was developed by addition of TMB Microwell Peroxidase Substrate (Kirkegaard and Perry, Gaithersburg, Md.), 50 μl per well, and stopped by the addition of 0.09 M sulfuric acid, 50 μl per well. Phosphotyrosine is estimated by measurement of absorbance at 450 nm. Signal from control wells containing no compound stimulated with EGF after subtraction of the background from wells without EGF was defined as 100% of control. Examination of extracts from these EGF stimulated cells by Western blotting with anti-phosphotyrosine indicated that the majority of the protein phosphotyrosine represented autophosphorylated EGFR or EGFR/erbB2 chimera respectively, but other protein substrates also displayed increased tyrosine phosphorylation. EGF typically increased total phosphotyrosine levels by approximately 4-fold in each transfected cell. IC₅₀ values represent the concentration of compound required to reduce the signal to 50% of control and were determined graphically from titrations over a 100-fold concentration range. Analysis of erbB Phosphorylation by Immunoprecipitation Followed by Western Blotting. SKBr3 cells were treated with compound or activating ligand as indicated. The media was aspirated, and 1 ml/75 cm² flask ice-cold immunoprecipitation lysis buffer (1.0% TX100; 10 mM Tris; 5 mM EDTA; 50 mM NaCl; 30 mM sodium orthovanadate with freshly added 100 μM PMSF, and 1 Complete™ protease inhibitor tablet (Roche Diagnostics, Indianapolis, Ind. per 50 ml buffer) was added. Immunoprecipitation was performed on 100 μl of lysate: EGFr was immunoprecipitated using Santa Cruz SC-120, 2 μl/100 μl lysate; erbB2 using Oncogene OP15, 1 μg/100 μl lysate; and erbB3 with Santa Cruz SC-285, 2 μl/100 μl lysate. All immunoprecipitations were carried out at 4° C. overnight, with rocking, in the presence of 30 μl of protein A beads. The beads with immobilized protein were isolated by centrifugation at 14,000 rpm, 4° C. for 10 seconds. The supernatants were aspirated and the pellets washed 3× with PBS with 0.1% Tween 20. The samples were then resuspended in 40 μl Laemmli buffer with DTT and boiled for 4 minutes. The samples were then loaded on a 4-12% PAGE. They were electrophoresed 1 hr at 150V using MES buffer. The gels were transferred to PVDF in the presence of 10% methanol. The membrane was blocked using blocking buffer (Roche Diagnostics, Indianapolis, Ind.) and the phosphotyrosine was detected using anti-PY54 antibody conjugated to horseradish peroxidase and developed by enhanced chemiluminescence according to the manufacturer's instructions (ECL™; Amersham, Pharmacia Biotech, Piscataway, N.J.; LumiGLO™; Cell Signaling). The signal was quantitated with a Lumi-imager™ (Boehringer Mannheim, Indianapolis, Ind.).

The following assay may also be employed for c-erbB2 kinase to determine the potency and selectivity of the compounds for their use as c-erbB2 inhibitors. The following assay is similar to that described previously in Schrang et. al. Anal. Biochem. 211, 1993, p233-239. Nunc MaxiSorp 96-well plates are coated by incubation overnight at 37° C. with 100 mL per well of 0.25 mg/mL Poly(Glu, Tyr) 4:1 (PGT) (Sigma Chemical Co., St. Louis, Mo.) in PBS (phosphate buffered saline). Excess PGT is removed by aspiration, and the plate is washed three times with wash buffer (0.1% Tween 20 in PBS). The kinase reaction is performed in 50 mL of 50 mM HEPES (pH 7.5) containing 125 mM sodium chloride, 10 mM magnesium chloride, 0.1 mM sodium orthovanadate, 1 mM ATP, 0.48 mg/mL (24 ng/well) c-erbB2 intracellular domain. The intracellular domain of the erbB2 tyrosine kinase (amino acids 674-1255) is expressed as a GST fusion protein in Baculovirus and purified by binding to and elution from glutathione coated beads. The compound in DMSO (dimethylsulfoxide) is added to give a final DMSO concentration of 2.5%. Phosphorylation was initiated by addition of ATP (adenosine triphosphate) and proceeded for 6 minutes at room temperature, with constant shaking. The kinase reaction is terminated by aspiration of the reaction mixture and subsequent washing with wash buffer (see above). Phosphorylated PGT is measured by 25 minutes of incubation with 50 mL per well HRP-conjugated PY54 (Oncogene Science Inc. Uniondale, N.Y.) antiphosphotyrosine antibody, diluted to 0.2 mg/mL in blocking buffer (3% BSA and 0.05% Tween 20 in PBS). Antibody is removed by aspiration, and the plate is washed 4 times with wash buffer. The colorimetric signal is developed by addition of TMB Microwell Peroxidase Substrate (Kirkegaard and Perry, Gaithersburg, Md.), 50 mL per well, and stopped by the addition of 0.09 M sulfuric acid, 50 mL per well. Phosphotyrosine is estimated by measurement of absorbance at 450 nm. The signal for controls is typically 0.6-1.2 absorbance units, with essentially no background in wells without the PGT substrate and is proportional to the time of incubation for 10 minutes. Inhibitors are identified by reduction of signal relative to wells without inhibitor and IC₅₀ values corresponding to the concentration of compound required for 50% inhibition are determined. The compounds exemplified herein which correspond to formula 1 have IC₅₀ values of <10 mM against erbB2 kinase. IC₅₀ values may be used to determine selectivity by any means known in the art. For example, the ratio for IC₅₀ values at erbB1 receptors and erbB2 receptors (IC₅₀ erbB1≈IC₅₀ erbB2) can be used. Advantageously, the ratio exceeds two.

The in vivo anti-tumor activity of the compounds for use in the methods of the present invention can be determined by the amount of inhibition of tumor growth by a test compound relative to a control. The tumor growth inhibitory effects of various compounds can be measured according to the method of Corbett T. H., et al., “Tumor Induction Relationships in Development of Transplantable Cancers of the Colon in Mice for Chemotherapy Assays, with a Note on Carcinogen Structure”, Cancer Res., 35, 2434-2439 (1975) and Corbett T. H., et al., “A Mouse Colon-tumor Model for Experimental Therapy”, Cancer Chemother. Rep. (Part 2)”, 5, 169-186 (1975), with slight modifications. Tumors can be induced in the left flank of mice by subcutaneous (sc) injection of 1-5 million log phase cultured tumor cells suspended in 0.1 ml RPMI 1640 medium. After sufficient time has elapsed for the tumors to become palpable (˜100-150 mm³ in size/5-6 mm in diameter) the test animals (athymic female mice) are treated with test compound (formulated at a concentration of 10 to 15 mg/ml in 5 Gelucire or 0.5% methyl cellulose) by the intravenous (iv) or oral (po) route of administration once or twice daily for 7 to 29 consecutive days. In order to determine an anti-tumor effect, the tumor is measured in millimeters with a Vernier caliper across two diameters and the tumor size (mm³) is calculated using the formula: Tumor size (mm³)=(W×W)/2×L (L=length and W=width), according to the methods of Geran, R. I., et al. “Protocols for Screening Chemical Agents and Natural Products Against Animal Tumors and Other Biological Systems”, Third Edition, Cancer Chemother. Rep., 3, 1-104 (1972). Results are expressed as percent inhibition, according to the formula: Inhibition Growth (%)=[100-{(% Growth of Treated/% Growth of Control)×100}]. The flank site of tumor implantation provides reproducible dose/response effects for a variety of chemotherapeutic agents, and the method of measurement (tumor diameter) is a reliable method for assessing tumor growth rates.

Administration of erbB2 inhibitors can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of 0.001 to 200 mg per kg body weight per day, preferably 1 to 35 mg/kg/day. For a 70 kg human, this would amount to 0.05 to 7 g/day, preferably 0.2 to 2.5 g/day. In some instances, dosage levels below the lower limit of the aforesaid range may be more than adequate, while in other cases still larger doses may be employed without causing any harmful side effect.

The erbB2 inhibitors of the present invention may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the methods of the present invention. It is to be understood that the scope of the present invention is not limited in any way by the scope of the following examples and preparations.

The “test compound” used in the following Examples, unless otherwise indicated, is the selective erbB2 inhibitor, E-2-Methoxy-N-(3-{4-(3-methyl-4-(6-methyl-pyridin-3-yloxy)-phenylamino)-quinazolin-6-yl}-allyl)-acetamide.

### EXAMPLE 1

### The FRE Model: Effect of the Duration of Exposure on Anti-Tumor Efficacy of a Test Compound

An objective of the pre-clinical investigations was to determine whether the Cₘₐₓ or area under the curve (AUC) of the test compound is critical for the anti-tumor efficacy. An additional goal was to establish a pharmacokinetics/pharmacodynamics (PK/PD) relationship in the FRE/erbB2 tumor model. The FRE/erbB2 is an engineered murine tumor model, which over-expresses human erbB2 with a trans-membrane mutation.

The role of duration of the test compound exposure on FRE/erbB2 tumor growth in athymic mice was determined. The test compound was either administered using tail vein infusion or orally. Using tail vein infusion a calculated fixed Cₘₐₓ (1200 ng/ml) concentration was maintained during daily infusion while the duration of exposure and therefore AUC was varied. Treatments and plasma concentrations in treated animals is shown in Table 1.

A 1.15 mg/ml solution of the test compound was infused IV at 550 μl/hr for 2 minute ramped infusions followed by 50 μl/hr for 15 min or 4 hour daily infusions. (Projection was based on Cl of the test compound). Athymic female mice bearing FRE/erbB2 tumors (˜100 mm³ in size) were treated with vehicle, the test compound orally or the test compound intravenously. Body weight changes and tumor measurements were obtained at regular intervals (Days 1, 3, 5, and 7). The study was carried out for 7 days. Plasma and tumor samples were isolated for PK and PD analysis at the termination of study. The results on anti-tumor efficacy, tumor volume, body weight changes, plasma concentration of the test compound as well as p-erbB2 (the phosphorylated form of erbB2 receptor) inhibition in control and test compound animals are shown in Table 1.

TABLE 1

| | Plasma | | | | |
|---|---|---|---|---|---|
| | Concentration | % p-erbB2 | Tumor vol. (mm³; Mean ± SE) | | % |
| Treatment | (ng/ml; Mean ± SE) | reduction | Day 1 | Day 7 | GI |
| Vehicle, | 00 | 00 | 110 ± 18 (23) | 801 ± 92 (24) | 00 |
| 10 ml/kg | | | | | |
| PO, QD | | | | | |
| Test | 1460 ± 170 | 34 | 113 ± 18 (21) | 531 ± 101 (22) | 54* |
| Compound, | (0.5 h) | | | | |
| 25 mg/kg | | | | | |
| PO, QD | | | | | |
| Vehicle, | 00 | 00 | 107 ± 22 (21) | 1142 ± 335 (21) | 00 |
| 218 μL/day IV, | | | | | |
| QD | | | | | |
| Test | 448 ± 141 | 48 | 121 ± 24 (23) | 749 ± 178 (24) | 34 |
| Compound, | | | | | |
| 1.4 mg/kg IV, | | | | | |
| QD; 15 min/day | | | | | |
| Test | 473 ± 141 | 53 | 117 ± 23 (22) | 273 ± 81 (22) | 76 |
| Compound, | | | | | |
| 10.7 mg/kg IV; | | | | | |
| 4 hr/day | | | | | |

Values in parenthesis are the average body weight (g);
*Compare to Vehicle (IV) group
PO, QD study N = 6;
IV, QD study N = 4
% GI = % Growth Inhibition

Approximately 54% tumor growth inhibition was achieved in animals treated with daily oral administration of the test compound. Plasma concentration at 0.5 hr post-dosing on day 7 was 1460 ng/ml. The test compound treatments were safe and did not cause any body weight loss or mortality.

Daily 15-minute infusion of the test compound resulted in approximately 34% growth inhibition. In contrast, equivalent infusion for 4 hr/day resulted in substantially higher tumor growth inhibition (76%). This suggests that the duration of coverage above a threshold plasma concentration has a significant value in the overall anti-tumor efficacy of the test compound in this model. Based on these results, it can be also concluded that the coverage (AUC) for 4 hr/day at an approximate plasma concentration of 500 ng/ml is sufficient to cause substantial FRE/erbB2 tumor growth inhibition. The duration of exposure or AUC (Coverage) significantly affect efficacy: the daily Cₘₐₓ alone cannot explain efficacy in this model.

The duration of coverage (˜4 hr/day) at a plasma concentration of ˜500 ng/ml has an advantage over a shorter duration of coverage (˜15 min/day) in the FRE/erbB2 tumor model.

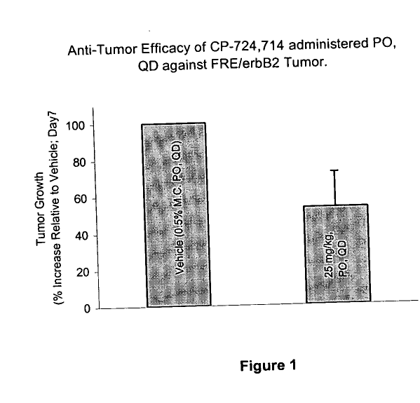

The anti-tumor efficacy of 25 mg/kg of the test compound administered orally once a day was effective at slowing volume growth of the FRE tumors in the nu/nu mice is shown in bar graph format in FIG. 1. The figure shows that at seven days of treatment the FRE tumor volume in treated mice is about half of the control.

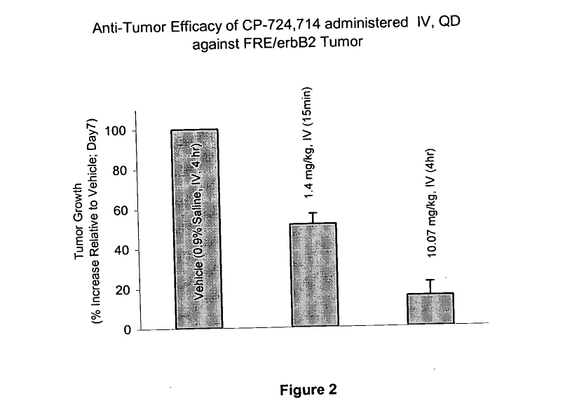

FIG. 2 shows in bar graph format that the anti-tumor efficacy of the 10 mg/kg of the test compound administered IV for seven days over a four hour period each day is highly effective both on an absolute basis and when compared to infusion of either about 1.4 mg/kg of the inhibitor daily over about 15 min/day or vehicle. The test compound at about 10 mg/kg slowed the tumor volume increase to less than 24% of the vehicle control. By contrast, rapid infusion of about 1.4 mg/kg slowed the tumor volume increase to less than 66% of the vehicle control.

### EXAMPLE 2

### The SK-OV-3 Model: Effect of the Duration of Exposure on Anti-Tumor Efficacy of the Test Compound

Pre-clinical investigations were conducted to determine whether the duration of the test compound coverage is critical for the anti-tumor efficacy. Another goal was to establish the minimum efficacious (Cₘₐₓ and Cave₀₋₄ h) concentration in human ovarian adenocarcinoma, SK-OV-3 tumor model.

As background, the test compound (PO, QD) was shown in Example 1 to be efficacious against FRE erbB2 tumors. Similarly, IV administration of test compound was efficacious against FRE erbB2 tumors. The findings demonstrated that maintaining ˜500 ng/ml blood concentrations of the test compound for 4 hr/day has an advantage over a shorter duration of coverage (˜15 min/day) with comparable p-erbB2 reduction (48-53%) in the FRE erbB2 tumor model. Pharmacokinetic, pharmacodynamic and efficacy data are shown in Table 1.

Based on the exposure measured in earlier studies, a Cₘₐₓ of ˜1200 ng/ml or AUC₀₂ h of ˜985 ng·hr/ml for the test compound with coverage of ˜2 hours was critical for ˜50% FRE erbB2 tumor growth inhibition.

The investigation was extended to the human xenograft model, human ovarian adenocarcinoma model SK-OV-3, which over-expresses erbB2.

SK-OV-3 cells obtained from ATCC (Rockville, Md.) were grown in McCoy's medium containing 10% fetal bovine serum and pen/strep. Exponentially growing cells were harvested and inoculated SC (5 million cells/animal) into female athymic mice. Athymic mice bearing SK-OV-3 tumors (˜100 mm³ in size) were randomized in 7 groups as shown in Table 2. The tumor measurements and body weight changes were obtained on days 1, 3, 6, 10, 13 and 18. Tumor volume was calculated by the following formula: Tumor volume (mm³)═(W×W)/2×L (L=length & W=width). Blood samples (˜50 μl) were isolated at 0.5, 1, 2, 4 and 8 hrs after dosing on day 18 for PK-analysis. Tumors were isolated at 0.5 hr post-dosing on day 18 for PD-analysis by ELISA. The p-erbB2 reduction, tumor volume and body weight changes in control and test compound treated animals are shown below in Table 2.

TABLE 2

| | | | | % |
|---|---|---|---|---|
| | | Tumor volume | | Growth |
| | % p-erbB2 | (mm³; Mean ± SE) | | inhibi- |
| Treatment | reduction | Day 1 | Day 18 | tion |
| Vehicle, 10 ml/kg | 00 | 99 ± 15 (24) | 398 ± 53 (25) | 00 |
| PO, BID | | | | |
| Test Compound, | 14 | 98 ± 14 (23) | 390 ± 38 (24) | 2 |
| PO, QD | | | | |
| 50 mg/kg | | | | |
| (Total Daily | | | | |
| Dose = 50 mg/kg) | | | | |
| Test Compound, | 75 | 97 ± 14 (23) | 306 ± 36 (25) | 23 |
| PO, QD | | | | |
| 100 mg/kg | | | | |
| (Total Daily | | | | |
| Dose = 100 mg/kg) | | | | |
| Test Compound, | 90 | 98 ± 14 (23) | 254 ± 39 (24) | 36 |
| PO, QD | | | | |
| 200 mg/kg | | | | |
| (Total Daily | | | | |
| Dose = 200 mg/kg) | | | | |
| Test Compound, | 20 | 93 ± 12 (24) | 281 ± 42 (26) | 29 |
| PO, BID | | | | |
| 25 mg/kg | | | | |
| (Total Daily | | | | |
| Dose = 50 mg/kg) | | | | |
| Test Compound, | 24 | 94 ± 13 (24) | 218 ± 38 (25) | 45 |
| PO, BID | | | | |
| 50 mg/kg | | | | |
| (Total Daily | | | | |
| Dose = 100 mg/kg) | | | | |
| Test Compound, | 62 | 94 ± 13 (23) | 115 ± 24 (23) | 71 |
| PO, BID | | | | |
| 100 mg/kg | | | | |
| (Total Daily | | | | |
| Dose = 200 mg/kg) | | | | |

Values in parenthesis are the average body weight (g).

TABLE 3
Pharmacokinetics of the test compound in SK-OV-3 tumor-bearing mice

| | Cₘₐₓ 0.5 h | AUC₀₋₄ h | Cave₀₋₄ h |
|---|---|---|---|
| Groups | (ng/ml) | (ng-hr/ml)* | (ng/ml) |
| 50 mg/kg, PO, QD | 3640 | 3410 | 853 |
| 100 mg/kg, PO, QD | 12100 | 16300 | 4080 |
| 200 mg/kg, PO, QD | 10200 | 15100 | 3780 |
| 25 mg/kg, PO, BID | 1780 | 1560 | 390 |
| 50 mg/kg, PO, BID | 3880 | 4180 | 1050 |
| 100 mg/kg, PO, BID | 8060 | 9330 | 2330 |

Values represent the average.
*No significant difference was observed between AUC₀₋ₜₗₐₛₜ and AUC₀₋₄ h.

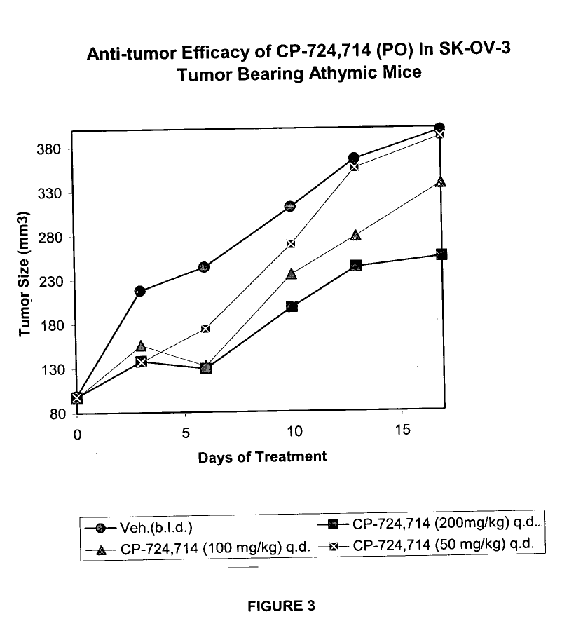

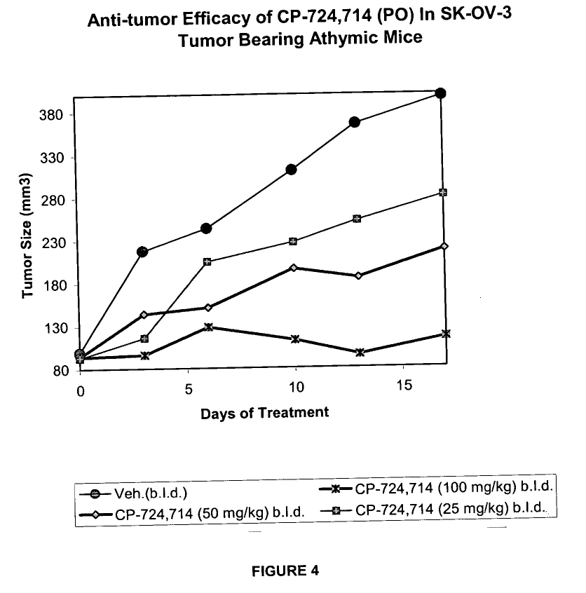

Oral anti-tumor efficacy of the test compound (QD and BID) was determined against human ovarian adenocarcinoma model SK-OV-3 which overexpresses erbB2. Moreover, the test compound administration (QD or BID) was efficacious and caused dose-dependent inhibition of SK-OV-3 xenografts (FIGS. 3 and 4). The test compound was well tolerated and there was no body weight loss or animal mortality.

The QD dosing of the test compound at 50 mg/kg for 18 days was non-efficacious. Approximately 29% tumor growth inhibition was achieved when a total daily dose of 50 mg/kg/day was administered on a BID schedule (25 mg/kg, BID). The reduction of erbB2 receptor autophosphorylation at 0.5 hr post-dosing on day 18 was comparable in both QD and BID treatment groups (14-20%), however, the Cₘₐₓ for the test compound in 50 mg/kg QD group was approximately 2-fold higher compared to 25 mg/kg BID dosed animals (Cₘₐₓ, 3640 ng/ml vs. 1780 ng/ml). Similarly, the AUC₀₋₄ h (3410 ng.hr/ml vs. 1560 ng.hr/ml) and Cave₀₋₄ h (853 ng/ml vs. 390 ng/ml) in QD group was approximately 2-fold higher compared to BID dosed group. These results demonstrate that neither higher Cₘₐₓ nor AUCO₄ h are critical for the anti-tumor efficacy of the test compound. An average coverage of 390 ng/ml of the test compound (Cave₀₋₄ hr) twice a day (BID) has a benefit over an average coverage of 853 ng/ml (Cave₀₋₄ hr) once a day (QD) though both approaches (QD & BID) gave comparable reduction of erbB2 autophosphorylation.

The benefit of BID over QD dosing was also observed at higher doses of the test compound in the SK-OV-3 model. In comparison with 50 mg/kg BID dosing of the test compound (100 mg/kg/day), QD dosing of 100 mg/kg/day resulted in higher reduction of erbB2-autophosphorylation (75% vs. 24%) and was associated with higher Cₘₐₓ (12,100 ng/ml vs. 3880 ng/ml), AUCO₄ h (16,300 ng.hr/ml vs. 4180 ng.hr/ml) and Cave₀₋₄ h (4080 ng/ml vs. 1050 ng/ml). However, the QD schedule was less efficacious than the BID schedule (23% vs. 45% tumor growth inhibition). These results support the interpretation that higher Cₘₐₓ, or AUC₀₋₄ h of the test compound does not have any significant benefit in this tumor model whereas the frequency of coverage (Cave₀₄, BID versus QD) above a threshold level is the determining factor for the anti-tumor efficacy. Furthermore, an approximately 24% reduction of SK-OV-3 tumor p-erbB2 may be sufficient for ˜50% growth inhibition if the average duration of coverage is maintained for a longer period of time with BID dosing.

Oral absorption of the test compound was non-linear at 200 mg/kg QD dosing. The Cₘₐₓ and the Cave₀₋₄ h values for the test compound were comparable in both 200 mg/kg QD and 100 mg/kg BID dosed animals. Despite the lower reduction of tumor erbB2-autophosphorylation in 100 mg/kg BID dosed animals (62% vs. 90%), the tumor growth inhibition in this group was 2-fold higher than 200 mg/kg, QD dosed animals (71% vs. 36%). These observations further support the interpretation that a lower reduction of erbB2-autophosphorylation (62% vs. 90%) with a longer/more frequent daily coverage (BID schedule) at a comparable Cₘₐₓ has significant benefit.

The present findings are in accord with the results in athymic mice bearing FRE erbB2 tumors (Example 1). In that study, compared to 15 min/day, maintaining ˜500 ng/ml blood concentrations of the test compound for 4 hr/day with a comparable reduction of erbB2-autophosphorylation had a benefit.

Thus, in this example, the findings of SK-OV-3 tumor model suggest that the total daily coverage, i.e. frequency of daily dosing, is critical for the anti-tumor efficacy of the test compound. That is, a BID schedule has a benefit over QD dosing. The higher reduction of erbB2-autophosphorylation for a shorter duration has limited value.

### EXAMPLE 3

### Effect of the Duration of Exposure on Anti-Tumor Efficacy of the Test Compound

Pre-clinical investigations were conducted to determine whether the duration of the test compound coverage is critical for the anti-tumor efficacy and also to establish the minimum efficacious (Cₘₐₓ and Cave₀₋₄ h) concentration in the human breast adenocarcinoma, BT-474 tumor model.

As background, the test compound (PO, QD) was shown in Example 1 to be efficacious against FRE erbB2 tumors. Similarly, IV administration of test compound was efficacious against FRE erbB2 tumors. The findings demonstrated that maintaining ˜500 ng/ml blood concentrations of the test compound for 4 hr/day has an advantage over a shorter duration of coverage (˜15 min/day) with comparable p-erbB2 reduction (48-53%) in the FRE erbB2 tumor model. Pharmacokinetic, pharmacodynamic and efficacy data are shown in Table 1.

Based on the exposure measured in the earlier study in FRE erbB2 model the investigation was extended in Example 2 to the human ovarian adenocarcinoma xenograft model SK-OV-3, which overexpresses erbB2. The test compound was efficacious and the findings of the SK-OV-3 tumor model suggested that the total daily coverage, i.e. frequency of daily dosing, is critical for the anti-tumor efficacy of the test compound. A BID dosing schedule is more beneficial than a QD dosing schedule. The higher reduction of erbB2-autophosphorylation for a shorter duration has limited value.

The present example extends the evaluation of the significance of the frequency of daily dosing for the anti-tumor efficacy of the test compound to a human breast adenocarcinoma model BT-474, which over-expresses erbB2 receptors.

Exponentially growing BT-474 cells (RPMI 1640 with 10 mM HEPES, 10% FBS, and pen/strep [Gibco]) were harvested and inoculated SC (5 million cells/animal) into female athymic mice. Trochar pieces of BT-474 tumors were then implanted into the right flank of animals. BT-474 tumor bearing mice (50-320 mm³ in size, N=40) were randomized in 7 groups consisting 5-6 animals each. Animals were treated with vehicle (PO, BID) or the test compound (PO, QD or BID) as described in Table 4. The tumor measurements and body weight changes were obtained on days 1, 6, 11, 15 and 22. Tumor volume was calculated by the following formula: Tumor volume (mm³)═(W×W)/2×L (L=length & W=width). Blood samples (˜50 μl) were isolated at 0.5, 1, 2, 4 and 8 hrs after dosing on day 22 for PK-analysis. Tumors were isolated at 0.5 hr post-dosing on day 22 for PD-analysis by ELISA.

Statistical Analysis: ANOVA was conducted on the percentage growth data and planned comparisons were conducted between like-doses. The data were log transformed for the analysis due to the distribution of the values. The Dunnett-Tamahane procedure was used for the multiple comparison analysis. The p-erbB2 reduction, tumor volume and body weight changes in control and test compound treated animals is shown in Table 4.

TABLE 4

| | | | | % |
|---|---|---|---|---|
| | | Tumor volume | | Growth |
| | % p-erbB2 | (mm³; Mean ± SE) | | inhibi- |
| Treatment | reduction | Day 1 | Day 22 | tion |
| Vehicle, | 00 | 113 ± 16 (25) | 701 ± 144 (30) | 00 |
| 10 ml/kg PO, | | | | |
| BID | | | | |
| Test Compound, | No | 78 ± 18 (25) | 376 ± 79 (29) | 22 |
| PO, QD, | detectable | | | |
| 15 mg/kg | reduction | | | |
| (Total Daily | | | | |
| Dose = 15 mg/kg) | | | | |
| Test Compound, | 57 | 139 ± 31 (23) | 635 ± 189 (27) | 33 |
| PO, QD, | | | | |
| 30 mg/kg | | | | |
| (Total Daily | | | | |
| Dose = 30 mg/kg) | | | | |
| Test Compound, | 75 | 153 ± 40 (25) | 608 ± 136 (29) | 35 |
| PO, QD, | | | | |
| 50 mg/kg | | | | |
| (Total Daily | | | | |
| Dose = 50 mg/kg) | | | | |
| Test Compound, | No | 114 ± 47 (24) | 520 ± 254 (29) | 54 |
| PO, BID, | detectable | | | |
| 15 mg/kg | reduction | | | |
| (Total Daily | | | | |
| Dose = 30 mg/kg) | | | | |
| Test Compound, | 26 | 161 ± 44 (26) | 530 ± 240 (30) | 68 |
| PO, BID, | | | | |
| 30 mg/kg | | | | |
| (Total Daily | | | | |
| Dose = 60 mg/kg) | | | | |
| Test Compound, | 74 | 155 ± 42 (24) | 413 ± 98 (28) | 68 |
| PO, BID, | | | | |
| 50 mg/kg | | | | |
| (Total Daily | | | | |
| Dose = | | | | |
| 100 mg/kg) | | | | |

Values in parenthesis are the average body weight (g).

The pharmacokinetics of the test compound in BT-474 tumor-bearing mice is shown in Table 5.

TABLE 5

| | | | |
|---|---|---|---|
| | Cₘₐₓ 0.5 h | AUC₀₋₄ h | |
| Groups | (ng/ml) | (ng · hr/ml) | Cave₀₋₄ h (ng/ml) |

| | | | |
|---|---|---|---|
| 15 mg/kg, PO, QD | 250 | Nd | nd |
| 30 mg/kg, PO, QD | 1800 | 1280* | 320* |
| 50 mg/kg, PO, QD | 5890 | 4220* | 1060* |
| 15 mg/kg, PO, BID | 616 | 480 | 120 |
| 30 mg/kg, PO, BID | 1570 | 1440* | 360* |
| 50 mg/kg, PO, BID | 6170 | 5280 | 1320 |

nd: not determined due to the extrapolated portion of AUC ≧ 30% of total AUC Values represent the average.
*Values were estimated based on the extrapolated concentration at 4 hr from 2 hr and 8 hr exposures.

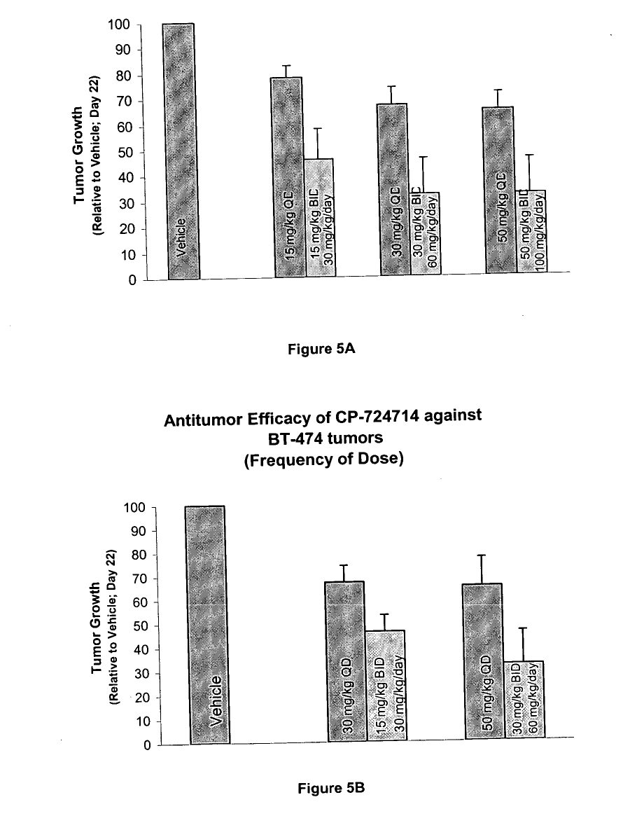

Thus, oral anti-tumor efficacy of the test compound (QD and BID) was determined against human breast adenocarcinoma model BT-474 which overexpresses erbB2. The test compound administration (QD or BID) was efficacious and caused growth inhibition of BT-474 xenografts (FIGS. 5a and 5b). The test compound was well tolerated and there was no body weight loss or animal mortality. Due to a wide variation in the initial tumor volume, % growth of individual tumor was calculated and an average of each group was used to determine relative anti-tumor efficacy.

The test compound treatments at 15 mg/kg QD (15 mg/kg/day) and BID (30 mg/kg/day) for 22 days were efficacious and caused 22% and 54% (p=0.007) tumor growth inhibition, respectively. The reduction of erbB2 receptor autophosphorylation at 0.5 hr post-dosing on day 22 was below the limit of detection in both QD and BID treatment groups and the determination of Cave₀₋₄ h in QD dosed animals was not possible due to the extrapolated portion of AUC≧30% of total AUC. The efficacious Cₘₐₓ, AUC₀₋₄ h and Cave₀₋₄ h (54% growth inhibition) for the test compound in 15 mg/kg, BID dosed animals were 616 ng/ml, 480 ng-hr/ml and 120 ng/ml, respectively.

The PK, PD and anti-tumor efficacy of the test compound was also determined after 30 mg/kg QD (30 mg/kg/day) and BID (60 mg/kg/day) treatments. The PK values were comparable for the test compound after QD or BID dosing determined on day 22 i.e. Cₘₐₓ (1800 ng/ml vs. 1570 ng/ml), AUCO₄ h (1280 ng·hr/ml vs. 1440 ng-hr/ml) and Cave₀₋₄ h (320 ng/ml vs. 360 ng/ml, Table 5). The reduction of BT-474 tumor erbB2 autophosphorylation in QD dosed animals was higher than BID dosed animals (57% vs. 26%, p=0.06). The 30 mg/kg BID schedule of the test compound was more efficacious than QD dosing (68% vs. 33% growth inhibition, p=0.053).

In comparison with 30 mg/kg QD or BID dosing of the test compound (30 mg/kg/day or 60 mg/kg/day), QD or BID dosing of 50 mg/kg/day (50 mg/kg/day or 100 mg/kg/day) resulted in greater reduction of tumor erbB2-autophosphorylation (˜75% reduction). The PK-parameters of the test compound in 50 mg/kg QD or BID treatment groups on day 22 were also comparable i.e. Cₘₐₓ (5890 ng/ml vs. 6170 ng/ml), AUCO₄ h (4220 ng-hr/ml vs. 5280 ng-hr/ml) and Cave₀₋₄ h (1060 ng/ml vs. 1320 ng/ml). The QD schedule appeared less efficacious than the BID schedule (35% vs. 68% tumor growth inhibition, p=0.066).

A pooled test, comparing like-doses between QD and BID, was performed. This test showed that, overall, the BID dosings were more efficacious than QD dosing (p=0.0346). This finding suggests that the multiplicity of the test compound-dosing has positive effect on overall outcome of treatment.

A comparison of PK, PD and anti-tumor efficacy of the test compound observed in 50 mg/kg, QD (50 mg/kg/day) vs. 30 mg/kg, BID (60 mg/kg/day) groups (the two closest groups in the total daily dosing) were also evaluated to determine the value of dosing-frequency. The p-erbB2 reduction in 50 mg/kg, QD (50 mg/kg/day) dosed group was much higher than 30 mg/kg, BID (60 mg/kg/day) dosed group (75% vs. 26% p-erbB2 reduction, Table 4). Similarly, higher Cₘₐₓ (5890 ng/ml vs. 1570 ng/ml), AUC₀₋₄ h (4220 ng·hr/ml vs. 1440 ng-hr/ml) and Cave₀₋₄ h (1060 ng/ml vs. 360 ng/ml) for the test compound was observed in 50 mg/kg, QD dosed group compared to 30 mg/kg, BID dosed group (Table 5). Despite the lower p-erbB2 reduction and PK-values for the test compound (i.e., Cₘₐₓ, AUC₀₋₄ h and Cave₀₋₄ h), 30 mg/kg, BID dosing (60 mg/kg/day) was more efficacious than 50 mg/kg, QD dosing (50 mg/kg/day). Overall, approximately 68% and 35% tumor growth inhibition was observed in 30 mg/kg, BID and 50 mg/kg, QD groups, respectively (p=0.0636). Although the total daily dose of the test compound in these two groups is slightly unequal, a conclusion can be made that the frequency of daily dosing i.e. BID dosing has benefit over QD dosing.

These results are similar to the findings with the SK-OV-3 tumor model study, Example 2, supra, that the frequency of daily dosing i.e. the Cave₀₋₄ twice a day coverage with BID dosing confers a benefit compared to Cave₀₋₄ once a day coverage with QD dosing. Furthermore, an approximately 26% reduction of BT-474 tumor-autophosphorylation twice a day with BID dosing may be sufficient for ˜50% growth inhibition if the average duration of coverage (˜360 ng/ml) is maintained for a longer period of time with BID dosing. The present findings are also in accord with the results of IV administration of the test compound by infusion into athymic mice bearing FRE erbB2 tumors. That study demonstrated that maintaining ˜500 ng/ml blood concentrations of the test compound for 4 hr/day conferred a benefit compared to a bolus administration.

Thus, the findings from the BT-474 tumor model suggest that both multiplicity of dosing and the frequency of daily dosing are critical for the anti-tumor efficacy of the test compound. Multiplicity of dosing relates to administering a dose (X mg/kg) from at least twice a day to six or optionally seven times per day compared to administering the same dose (X mg/kg) once per day. Frequency of daily dosing relates to dividing a daily dose, for example one half X mg/kg twice per day compared to X mg/kg once per day.

The higher reduction of erbB2-autophosphorylation for a shorter duration has limited value.

### EXAMPLE 4

Effect of the Duration of Exposure on Anti-Tumor Efficacy of the Test Compound

Pre-clinical investigations were conducted to determine whether the duration of the test compound coverage is critical for the anti-tumor efficacy and also to establish the minimum efficacious (Cₘₐₓ and Cave₀₋₄ h) concentration in the human breast adenocarcinoma tumor model, MDA-MB-453.

As background, the test compound (PO, QD) was shown in Example 1 to be efficacious against FRE erbB2 tumors. Similarly, IV administration of test compound was efficacious against FRE erbB2 tumors. The findings demonstrated that maintaining ˜500 ng/ml blood concentrations of the test compound for 4 hr/day has an advantage over a shorter duration of coverage (˜15 min/day) with comparable p-erbB2 reduction (48-53%) in the FRE erbB2 tumor model. Pharmacokinetic, pharmacodynamic and efficacy data are shown in Table 1.

The investigation was extended to the human ovarian adenocarcinoma xenograft model SK-OV-3 which overexpresses erbB2. The test compound was efficacious and the findings of SK-OV-3 tumor model suggest that the total daily coverage, i.e. frequency of daily dosing is critical for the anti-tumor efficacy of the test compound (BID schedule has benefit over QD dosing). The anti-tumor effect of QD vs. BID oral dosing schedules of the test compound was also investigated against the BT-474 human breast adenocarcinoma model which overexpresses erbB2. The findings also suggest that both multiplicity and frequency of dosing are critical for the anti-tumor efficacy of the test compound. Overall, the findings of both SK-OV-3 and BT-474 models suggest that the higher reduction of erbB2-autophosphorylation for a shorter duration has limited value.

The present investigation was performed to determine the oral anti-tumor efficacy of the test compound against an additional human breast carcinoma model, MDA-MB-453 which overexpresses erbB2. Our second objective of this investigation was to determine whether multiplicity or frequency of the test compound dosing has any benefit against this model.

Study Design: Exponentially growing MDA-MB-453 cells (DMEM/F12 with 10% FBS, and pen/strep [Gibco]) were harvested and inoculated SC (5 million cells/animal) into female athymic mice. MDA-MB-453 tumor bearing mice (˜100 mm³ in size, N=64) were randomized in 8 groups consisting 8 animals each. Animals were treated with vehicle (PO, QD or BID) or the test compound (PO, QD or BID) as described in Table 6. The tumor measurements and body weight changes were obtained on days 1, 3, 7, 10, 14, 17, 21, 24, and 29. Tumor volume was calculated by the following formula: Tumor volume (mm³)═(W×W)/2×L (L=length & W=width). Blood samples (˜50 μl) were isolated at 0.5, 1, 2, 4 and 8 hrs after dosing on day 29 for PK-analysis. Tumors were isolated at 0.5 hr post-dosing on day 29 for PD-analysis by ELISA.

Statistical Analysis: ANOVA was conducted on the percentage growth data and planned comparisons were conducted between like-doses. The data were log transformed for the analysis due to the distribution of the values. The Dunnett-Tamahane procedure was used for the multiple comparison analysis.

The p-erbB2 reduction, tumor volume and body weight changes in control and test compound treated animals are shown in Table 6.

TABLE 6

| | | | | % |
|---|---|---|---|---|
| | | Tumor volume | | Growth |
| | % p-erbB2 | (mm³; Mean ± SE) | | inhibi- |
| Treatment | reduction | Day 1 | Day 29 | tion |
| Vehicle, 10 ml/kg | 00 | 107 ± 5 (22) | 284 ± 19 (26) | 00 |
| PO, QD | | | | |
| Test Compound, | 78 | 107 ± 4 (23) | 213 ± 19 (25) | 38 |
| PO, QD 50 mg/kg | | | | |
| (Total Daily | | | | |
| Dose = 50 mg/kg) | | | | |
| Test Compound, | 88 | 107 ± 4 (23) | 175 ± 14 (25) | 63 |
| PO, QD 100 mg/kg | | | | |
| (Total Daily | | | | |
| Dose = 100 mg/kg) | | | | |
| Test Compound, | 92 | 107 ± 4 (22) | 108 ± 9 (24) | 100 |
| PO, QD 200 mg/kg | | | | |
| (Total Daily | | | | |
| Dose = 200 mg/kg) | | | | |
| Vehicle, 10 ml/kg, | 00 | 107 ± 4 (23) | 284 ± 20 (25) | 00 |
| PO, BID | | | | |
| Test Compound, | 69 | 107 ± 4 (22) | 252 ± 24 (23) | 19 |
| PO, BID 25 mg/kg | | | | |
| (Total Daily | | | | |
| Dose = 50 mg/kg) | | | | |
| Test Compound, | 75 | 107 ± 4 (23) | 164 ± 13 (24) | 66 |
| PO, BID 50 mg/kg | | | | |
| (Total Daily | | | | |
| Dose = 100 mg/kg) | | | | |
| Test Compound, | 79 | 107 ± 4 (23) | 137 ± 6 (25) | 83 |
| PO, BID 100 mg/kg | | | | |
| (Total Daily | | | | |
| Dose = 200 mg/kg) | | | | |

Values in parenthesis are the average body weight (g).

Pharmacokinetics of the test compound in MDA-MB-453 tumor-bearing mice are shown in Table 7.

TABLE 7

| | Cₘₐₓ 0.5 h | AUC₀₋₄ h | |
|---|---|---|---|
| Groups | (ng/ml) | (ng · hr/ml) | Cave₀₋₄ h (ng/ml) |
| 50 mg/kg, PO, QD | 2760 | 2360 | 591 |
| 100 mg/kg, PO, QD | 9770 | 12500 | 3120 |
| 200 mg/kg, PO, QD | 16700 | 26100 | 6510 |
| 25 mg/kg, PO, | 952 | 857 | 215 |
| BID | | | |
| 50 mg/kg, PO, | 2390 | 2040 | 509 |
| BID | | | |
| 100 mg/kg, PO, | 6870 | 6840 | 1710 |
| BID | | | |

Values represent the average.

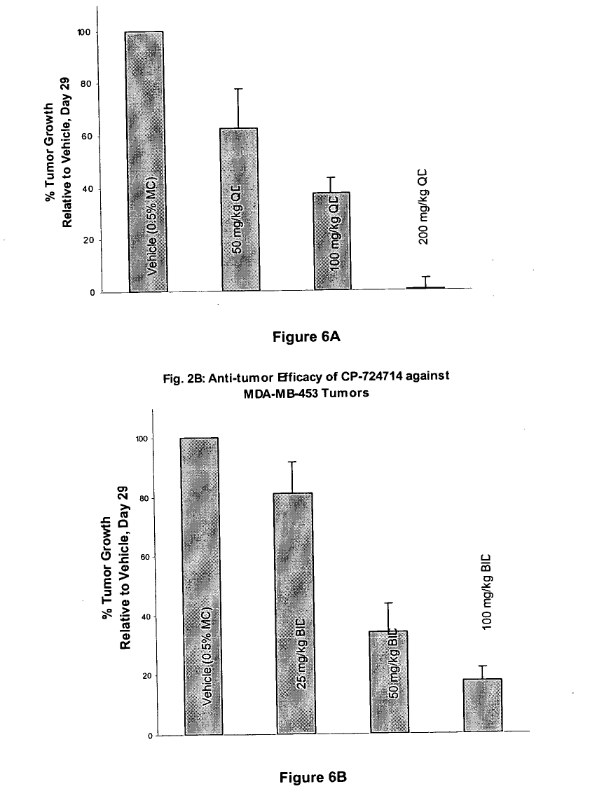

Thus, oral anti-tumor efficacy of the test compound (QD and BID) was determined against human breast adenocarcinoma model MDA-MB-453 which overexpresses erbB2. The test compound administration (QD or BID) was efficacious and caused growth inhibition of MDA-MB-453 xenografts (FIGS. 6a and 6b). The test compound was well tolerated and there was no body weight loss or animal mortality.

The test compound treatments at 50, 100 and 200 mg/kg QD (50, 100 and 200 mg/kg/day) for 29 days were efficacious and caused 38%, 63% and 100% tumor growth inhibition, respectively. The reduction of erbB2 receptor autophosphorylation at 0.5 hr post-dosing on day 29 in 50, 100 and 200 mg/kg groups were 78%, 88% and 92%, respectively. BID dosing of 25, 50 and 100 mg/kg the test compound for 29 days was efficacious against MBA-MB-453 tumors and caused 19%, 66% and 83% growth inhibition, respectively. The p-erbB2 reduction in these groups were 69%, 75% and 79%, respectively.

ANOVA was used for statistical analysis of overall efficacy for the different doses of the test compound. Dunnett-Tamahane's procedure was used for multiple comparisons to vehicle adjustments. The results show that there is no significant difference between 25 mg/kg BID and the 50 mg/kg QD (p=0.295), the 50 mg/kg BID and the 100 mg/kg QD (p=0.703) and the 100 mg/kg BID and the 200 mg/kg QD (p=0.117) dosing schedules of the test compound. Similarly, there was no significant difference between like doses i.e. 50 mg/kg BID vs. 50 mg/kg QD (p=0.13) and 100 mg/kg BID vs. 100 mg/kg QD (p=0.17). Comparative statistical evaluation using only the dose/dosing-schedule and anti-tumor efficacy observed in different groups is not sufficient to derive any definitive conclusion to address the question: whether BID schedule has any benefit over QD dosing of the test compound.

The reduction of p-erbB2 after QD (50-200 mg/kg) or BID (25-100 mg/kg) dosings was 69-92% and it was difficult to use it as a parameter for any further statistical data analysis. Hence, the data-analysis was extended using pharmacokinetic parameters i.e. Cₘₐₓ and Cave₀₋₄ h of the test compound.

The Caves₀₋₄ h of 591 ng/ml and 3120 ng/ml obtained after 50 mg/kg (50 mg/kg/day) and 100 mg/kg (100 mg/kg/day) QD dosing caused 38% and 63% tumor growth inhibition. Cave₀₋₄ h of 509 ng/ml obtained twice a day with 50 mg/kg BID dosing schedule resulted in 66% efficacy. The Cave₀₋₄ h of 509 ng/ml maintained for 8 hrs/day with BID dosing is not significantly different from maintaining Cave₀₋₄ h at 591 ng/ml (50 mg/kg QD dosing) or 3120 ng/ml (100 mg/kg QD dosing) for 4 hrs/day (p=0.13 & p=0.58, respectively). This can also be interpreted that maintaining 509 ng/ml average plasma concentration for 8 hrs/day has equal or better benefit compared to maintaining average plasma concentrations of 591 to 3120 ng/ml for 4 hrs/day. The Cₘₐₓ for the test compound in the 50 mg/kg QD and 50 mg/kg BID groups was comparable (2760 ng/ml vs. 2390 ng/ml) whereas the Cₘₐₓ in the 100 mg/kg, QD group was approximately 4-fold higher (9770 ng/ml). These results suggest that higher Cₘₐₓ or Cave₀₋₄ h alone has limited value when p-erbB2 reduction is comparable.

A comparison of Cₘₐₓ and Cave₀₋₄ h vs. anti-tumor efficacy of the test compound observed in the 100 mg/kg BID and 200 mg/kg QD groups was also performed. The Cₘₐₓ for the test compound in the 200 mg/kg QD group was 2.4-fold higher than that in the 100 mg/kg BID group (16700 ng/ml vs. 6870 ng/ml). Similarly Cave₀₋₄ h was 3.8-fold higher in the 200 mg/kg QD group compared to the 100 mg/kg BID group (6510 ng/ml vs. 1710 ng/ml). Despite the higher Cₘₐₓ and Cave₀₋₄ h, the overall efficacy of the test compound observed in with the 200 mg/kg QD dose was comparable to the anti-tumor efficacy observed with 100 mg/kg BID dosing (100% vs. 83%). This data further suggest that maintaining 8 hrs/day average plasma concentration at 1710 ng/ml (Cₘₐₓ, 6870 ng/ml) by 100 mg/kg BID dosing of the test compound is as beneficial as maintaining 6510 ng/ml (Cₘₐₓ, 16,700 ng/ml) average plasma concentration after 200 mg/kg QD dosing.

Thus, the findings here suggest that in the MDA-MB-453 tumor model, maintaining 8 hrs/day ˜509 ng/ml plasma concentration of the test compound (50 mg/kg, BID dosing) is as effective as maintaining 4 hrs/day average plasma concentrations of 591 to 3120 ng/ml (50-100 mg/kg QD dosing) in inhibiting tumor growth. Thus a low dose of the test compound given on BID schedule has benefit equal to the higher doses given on QD schedule.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated herein by reference in their entireties.

1. A method for treating overexpression of the erbB2 receptor in a mammal in need of such treatment, said method comprising:
(a) administering to said mammal a therapeutically effective amount of a first inhibitor of the erbB2 receptor; and
(b) subsequently administering to said mammal, after an interval comprising less than 24 hours, from one to six therapeutically effective amounts of a second inhibitor of the erbB2 receptor.

2. The method of claim 1, wherein one therapeutically effective amount of said second inhibitor of the erbB2 receptor is administered in step (b) of said method.

3. The method of claim 1, wherein the interval in step (b) of said method is less than 12 hours.

4. The method of claim 1, wherein the interval in step (b) of said method is less than 1 hour.

5. The method of claim 1 wherein the first inhibitor in (a) is the same as second inhibitor in (b).

6. The method of claim 1 wherein the first inhibitor in (a) is other than the second inhibitor in (b).

7. The method of claim 1 wherein the first inhibitor in (a) is synergistic with the second inhibitor in (b).

8. The method of claim 1 wherein the first inhibitor in (a), the second inhibitor in (b), or both, are an antagonist of the erbB2 receptor.

9. The method of claim 1 wherein the first inhibitor in (a), the second inhibitor in (b), are independently selected from small molecules and monoclonal antibodies.

10. The method of claim 1 wherein the first inhibitor in (a), the second inhibitor in (b), or both, or a mixture thereof, comprise a compound of the formula 1: 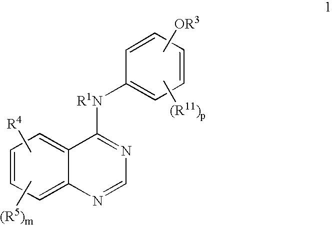
or a pharmaceutically acceptable salt, solvate or prodrug thereof, wherein:
m is an integer from 0 to 3;
p is an integer from 0 to 4;
each R¹ and R² is independently selected from H and C₁-C₆ alkyl;
R³ is —(CR¹R²)ₜ(4 to 10 membered heterocyclic), wherein t is an integer from 0 to 5, said heterocyclic group is optionally fused to a benzene ring or a C₅-C₈ cycloalkyl group, the —(CR¹R²)ₜ— moiety of the foregoing R³ group optionally includes a carbon-carbon double or triple bond where t is an integer between 2 and 5, and the foregoing R³ groups, including any optional fused rings referred to above, are optionally substituted by 1 to 5 R³ groups;
R⁴ is (CR¹⁶R¹⁷)ₘ—C═C(CR¹⁶R¹⁷)ₜR⁹, —(CR¹⁶R¹⁷)ₘC═C—(CR¹⁶R¹⁷X—R⁹, —(CR¹⁶R¹⁷)ₘ—C≡C—(CR¹⁶R¹⁷)ₖR¹³, —(CR¹⁶R¹⁷)ₘ—C═C—(CR¹⁶R¹⁷)ₖR¹³, or —(CR¹⁶R¹⁷)ₜR⁹, wherein the attachment point to R⁹ is through a carbon atom of the R⁹ group, each k is an integer from 1 to 3, each t is an integer from 0 to 5, and each m is an integer from 0 to 3;
each R⁵ is independently selected from halo, hydroxy, —NR¹R², C₁-C₆ alkyl, trifluoromethyl, C₁-C₆ alkoxy, trifluoromethoxy, —NR⁶C(O)R¹, —C(O)NR⁶R⁷, —SO₂NR⁶R⁷, —NR⁶C(O)NR⁷R¹, and —NR⁶C(O)OR⁷;
each R⁶, R^{6a} and R⁷ is independently selected from H, C₁-C₆ alkyl, —(CR¹R²)ₜ(C₆-C₁₀ aryl), and —(CR¹R²)ₜ(4 to 10 membered heterocyclic), wherein t is an integer from 0 to 5, 1 or 2 ring carbon atoms of the heterocyclic group are optionally substituted with an oxo(═O) moiety, the alkyl, aryl and heterocyclic moieties of the foregoing R⁶ and R⁷ groups are optionally substituted with 1 to 3 substituents independently selected from halo, cyano, nitro, —NR¹R², trifluoromethyl, trifluoromethoxy, C₁-C₆ alkyl, C₂-C₈ alkenyl, C₂-C₆ alkynyl, hydroxy, and C₁-C₆ alkoxy;
or R⁶ and R⁷, or R^{6a} and R⁷, when attached to the same nitrogen atom, can be taken together to form a 4 to 10 membered heterocyclic ring which may include 1 to 3 additional hetero moieties, in addition to the nitrogen to which said R⁶, R^{6a}, and R⁷ are attached, selected from N, N(R¹), O, and S, provided two O atoms, two S atoms or an O and S atom are not attached directly to each other;
each R⁸ is independently selected from oxo(═O), halo, cyano, nitro, trifluoromethoxy, trifluoromethyl, azido, hydroxy, C₁-C₆ alkoxy, C₁-C₁₀ alkyl, C₂-C₆ alkenyl, C₂-C₆ alkynyl, —C(O)R⁶, —C(O)OR⁶, —OC(O)R⁶, —NR⁶C(O)R⁷, —NR⁶SO₂NR⁷R¹, —NR⁶C(O)NR¹R⁷, —NR⁶C(O)OR⁷, —C(O)NR⁶R⁷, —NR⁶R⁷, —NR⁶OR⁷, —SO₂NR⁶R⁷, —S(O)ⱼ(C₁-C₆ alkyl) wherein j is an integer from 0 to 2, —(CR¹R²)ₜ(C₆-C₁₀ aryl), —(CR¹R²)ₜ(4 to 10 membered heterocyclic), —(CR¹R²)_{q}C(O)(CR¹R²)ₜ(C₆-C₁₀ aryl), —(CR¹R²)_{q}C(O)(CR¹R²)ₜ(4 to 10 membered heterocyclic), —(CR¹R²)ₜO(CR¹R²)_{q}(C₆-C₁₀ aryl), —(CR¹R²)ₜO(CR¹R²)_{q}(4 to 10 membered heterocyclic), —(CR¹R²)_{q}S(O)ⱼ(CR¹R²)ₜ(C₆-C₁₀ aryl), and —(CR¹R²)_{q}S(O)ⱼ(CR¹R²)ₜ(4 to 10 membered heterocyclic), wherein j is 0, 1 or 2, q and t are each independently an integer from 0 to 5, 1 or 2 ring carbon atoms of the heterocyclic moieties of the foregoing R⁸ groups are optionally substituted with an oxo(═O) moiety, and the alkyl, alkenyl, alkynyl, aryl and heterocyclic moieties of the foregoing R⁸ groups are optionally substituted with 1 to 3 substituents independently selected from halo, cyano, nitro, trifluoromethyl, trifluoromethoxy, azido, —OR⁶, —C(O)R⁶, —C(O)OR⁶, —OC(O)R⁶, —NR⁶C(O)R⁷, —C(O)NR⁶R⁷, —NR⁶R⁷, —NR⁶OR⁷, C₁-C₆ alkyl, C₂-C₆ alkenyl, C₂-C₆ alkynyl, —(CR¹R²)ₜ(C₆-C₁₀ aryl), and —(CR¹R²)ₜ(4 to 10 membered heterocyclic), wherein t is an integer from 0 to 5;
R⁹ is a non-aromatic mono-cyclic ring, a fused or bridged bicyclic ring, or a spirocyclic ring, wherein said ring contains from 3 to 12 carbon atoms wherein from 0 to 3 carbon atoms are optionally replaced with a hetero moiety independently selected from N, O, S(O)ⱼ wherein j is an integer from 0 to 2, and —NR¹—, provided that two O atoms, two S(O)ⱼ moieties, an O atom and a S(O)ⱼ moiety, an N atom and an S atom, or an N atom and an O atom are not attached directly to each other within said ring, and wherein the carbon atoms of said ring are optionally substituted with 1 or 2 R⁸ groups;
each R¹¹ is independently selected from the substituents provided in the definition of R⁸, except R¹¹ is not oxo(═O);
R¹² is R⁶, —OR⁶, —OC(O)R⁶, —OC(O)NR⁶R⁷, —OCO₂R⁶, —S(O)ⱼR⁶, —S(O)ⱼNR⁶R⁷, —NR⁶R⁷, —NR⁶C(O)R⁷, —NR⁶SO₂R⁷, —NR⁶C(O)NR^{6a}R⁷, —NR⁶SO₂NR^{6a}R⁷, —NR⁶CO₂R⁷, CN, —C(O)R⁶, or halo, wherein j is an integer from 0 to 2;
R¹³ is —NR¹R¹⁴ or —OR¹⁴;
R¹⁴ is H, R¹⁵, —C(O)R¹⁵, —SO₂R¹⁵, —C(O)NR¹⁵R⁷, —SO₂NR¹⁵R⁷, or —CO₂R¹⁵;
R¹⁵ is R¹⁸, —(CR¹R²)ₜ(C₆-C₁₀ aryl), —(CR¹R²)ₜ(4 to 10 membered heterocyclic), wherein t is an integer from 0 to 5, 1 or 2 ring carbon atoms of the heterocyclic group are optionally substituted with an oxo(═O) moiety, and the aryl and heterocyclic moieties of the foregoing R¹⁵ groups are optionally substituted with 1 to 3 R⁸ substituents;
each R¹⁸ and R¹⁷ is independently selected from H, C₁-C₆ alkyl, and —CH₂OH, or R¹⁶ and R¹⁷ are taken together as —CH₂CH₂— or —CH₂CH₂CH₂—;
R¹⁸ is C₁-C₆ alkyl wherein each carbon not bound to a N or O atom, or to S(O)ⱼ, wherein j is an integer from 0 to 2, is optionally substituted with R¹²;
and wherein any of the above-mentioned substituents comprising a CH₃ (methyl), CH₂ (methylene), or CH (methine) group, which is not attached to a halogeno, SO or SO₂ group or to a N, O or S atom, is optionally substituted with a group selected from hydroxy, halo, C₁-C₄ alkyl, C₁-C₄ alkoxy and —NR¹R².

11. The method of claim 1 wherein the first inhibitor in (a), the second inhibitor in (b), or both, or a combination thereof, comprise a compound selected from the group consisting of gefitinib (IRESSA, ZD1839), trastuzumab, cetuximab, erlotinib, IDM-1, ABX-EGF, canertinib hydrochloride, EGF-P64k vaccine, EKB-569, EMD-72000, GW-572016, MDX-210, ME-103, YMB-1001, 2C4 antibody, APC-8024, CP-724714, E75, Her-2/neu vaccine, Herzyme, TAK-165, ADL-681, B-17, D-69491, Dab-720, EGFrvIII, EHT-102, FD-137, HER-1 vaccine, HuMax-DGFr, ME-104, MR¹-1, SC-100, trastuzumab-DM1, YMB-1005, AEE-788 (Novartis), mTOR inhibitors, Rapamycin (Rapamune, Siolimus), CCI-779, AP23573 and RAD001.

12. The method of claim 1 further comprising achieving plasma levels of the first inhibitor in (a), the second inhibitor in (b), or both, between 10 ng/ml and 4000 ng/ml.

13. The method of claim 1 wherein the first inhibitor in (a) and the second inhibitor in (b) are each independently selected from the group consisting of:
(±)-(3-Methyl-4-(pyridin-3-yloxy)-phenyl)-(6-piperidin-3-ylethynyl-quinazolin-4-yl)-amine;
(±)-(3-Methyl-4-(pyridin-3-yloxy)-phenyl)-(6-piperidin-3-ylethynyl-quinazolin-4-yl)-amine;
(−)-(3-Methyl-4-(pyridin-3-yloxy)-phenyl)-(6-piperidin-3-ylethynyl-quinazolin-4-yl)-amine;
2-Methoxy-N-(3-{4-(3-methyl-4-(pyridin-3-yloxy)-phenylamino)-quinazolin-6-yl}-prop-2-ynyl)-acetamide;
(±)-(3-Methyl-4-(6-methyl-pyridin-3-yloxy)-phenyl)-(6-piperidin-3-ylethynyl-quinazolin-4-yl)-amine;
(±)-(3-Methyl-4-(6-methyl-pyridin-3-yloxy)-phenyl)-(6-piperidin-3-ylethynyl-quinazolin-4-yl)-amine;
(−)-(3-Methyl-4-(6-methyl-pyridin-3-ylethynyl-quinazolin-4-yl)-amine;
2-Methoxy-N-(3-{4-(3-methyl-4-(2-methyl-pyridin-3-yloxy)-phenylamino)-quinazolin-6-yl}-prop-2-ynyl)-acetamide;
(3-Methyl-4-(2-methyl-pyridin-3-yloxy)-phenyl)-(6-piperidin-4-ylethynyl-quinazolin-4-yl)-amine;
(3-Methyl-4-(6-methyl-pyridin-3-yloxy)-phenyl)-(6-piperidin-4-ylethynyl-quinazolin-4-yl)-amine;
2-Methoxy-N-(3-{4-(3-methyl-4-(6-methyl-pyridin-3-yloxy)-phenylamino)-quinazolin-6-yl}-prop-2-ynyl)-acetamide;
2-Fluoro-N-(3-{4-(3-methyl-4-(6-methyl-pyridin-3-yloxy)-phenylamino)-quinazolin-6-yl}-prop-2-ynyl)-acetamide;
E-2-Methoxy-N-(3-{4-(3-methyl-4-(6-methyl-pyridin-3-yloxy)-phenylamino)-quinazolin-6-yl}-allyl)-acetamide;
(3-Methyl-4-(pyridin-3-yloxy)-phenyl)-(6-piperidin-4-ylethynyl-quinazolin-4-yl)-amine;
2-Methoxy-N-(1-{4-(3-methyl-4-(6-methyl-pyridin-3-yloxy)-phenylamino)-quinazolin-6-ylethynyl}-cyclopropyl)-acetamide;
E-N-(3-{4-(3-Chloro-4-(6-methyl-pyridin-3-yloxy)-phenylamino)-quinazolin-6-yl}-allyl)-2-methoxy-acetamide;
N-(3-{(4-(3-Chloro-4-(6-methyl-pyridin-3-yloxy)-phenylamino)-quinazolin-6-yl}-prop-2-ynyl)-acetamide;
N-(3-{4-(3-Methyl-4-(6-methyl-pyridin-3-yloxy)-phenylamino)-quinazolin-6-yl}-prop-2-ynyl)-acetamide;
E-N-(3-{4-(3-Chloro-4-(6-methyl-pyridin-3-yloxy)-phenylamino)-quinazolin-6-yl}-allyl)-acetamide;
E-2-Ethoxy-N-(3-{4-(3-methyl-4-(6-methyl-pyridin-3-yloxy)-phenylamino)-quinazolin-6-yl}-allyl)-acetamide;
1-Ethyl-3-(3-{4-(3-methyl-4-(6-methyl-pyridin-3-yloxy)-phenylamino)-quinazolin-6-yl}-prop-2-ynyl)-urea;
Piperazine-1-carboxylic acid (3-{4-(3-methyl-4-(6-methyl-pyridin-3-yloxy)-phenylamino)-quinazolin-6-yl}-prop-2-ynyl)-amide;
(±)-2-Hydroxymethyl-pyrrolidine-1-carboxylic acid (3-{4-(3-methyl-4-(6-methyl-pyridin-3-yloxy)-phenylamino)-quinazolin-6-yl}-prop-2-ynyl)-amide;
(+)-2-Hydroxymethyl-pyrrolidine-1-carboxylic acid (3-{4-(3-methyl-4-(6-methyl-pyridin-3-yloxy)-phenylamino)-quinazolin-6-yl}-prop-2-ynyl)-amide;
(−)-2-Hydroxymethyl-pyrrolidine-1-carboxylic acid (3-{4-(3-methyl-4-(6-methyl-pyridin-3-yloxy)-phenylamino)-quinazolin-6-yl}-prop-2-ynyl)-amide;
2-Dimethylamino-N-(3-{4-(3-methyl-4-(pyridin-3-yloxy)-phenylamino)-quinazolin-6-yl}-prop-2-ynyl)-acetamide;
E-N-(3-{4-(3-Methyl-4-(6-methyl-pyridin-3-yloxy)-phenylamino)-quinazolin-6-yl}-allyl)-methanesulfonamide;
Isoxazole-5-carboxylic acid (3-{4-(3-methyl-4-(6-methyl-pyridin-3-yloxy)-phenylamino)-quinazolin-6-yl}-prop-2-ynyl)-amide;
1-(1,1-Dimethyl-3-{4-(3-methyl-4-(6-methyl-pyridin-3-yloxy)-phenylamino)-quinazolin-6-yl}-prop-2-ynyl)-3-ethyl-urea;
and the pharmaceutically acceptable salts, prodrugs and solvates of the foregoing compounds.

14. The method of claim 1 wherein the inhibitor is selected from the group consisting of: E-2-Methoxy-N-(3-{4-(3-methyl-4-(6-methyl-pyridin-3-yloxy)-phenylamino)-quinazolin-6-yl}-allyl)-acetamide; and pharmaceutically acceptable salts, prodrugs and solvates thereof.

15. A method of treating a subject having abnormal cell growth comprising orally, buccally, sublingually, intranasally, intraocularly, intragastrically, intraduodenally, topically, rectally, or vaginally administering to said subject in need of treatment for abnormal cell growth, within a twenty-four hour period, a first amount of an inhibitor of an erbB2 receptor, a therapeutically synergistically effective amount of a second inhibitor, and optionally, a third or fourth amount of said second inhibitor.
